(19) United States
(12) Patent Application Publication
(10) Pub. No.: US 2010/0093721 A1
(43) Pub. Date: Apr. 15, 2010

(54) PHENYL AND HETEROARYL SUBSTITUTED THIENO[2,3-d]PYRIMIDINES AND THEIR USE AS ADENOSINE A2a RECEPTOR ANTAGONISTS

(76) Inventors: J. Kent Barbay, Flourtown, PA (US); Devraj Chakravarty, Hillsborough, NJ (US); Kristi Leonard, Flourtown, PA (US); Brian Christopher Shook, Gilbertsville, PA (US); Aihua Wang, Jamison, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/479,247

(22) Filed: Jun. 5, 2009

Related U.S. Application Data
(60) Provisional application No. 61/104,785, filed on Oct. 13, 2008.

(51) Int. Cl. A61K 31/5377; C07D 495/04; A61K 31/519; A61P 25/16

(52) U.S. Cl. 514/234.2; 544/278; 514/260.1; 544/117

(57) ABSTRACT

This invention relates to a novel thieno[2,3-d]pyrimidine, Z, and its therapeutic and prophylactic uses, wherein X, R¹ and R² are defined in the specification. Disorders treated and/or prevented include Parkinson's Disease.

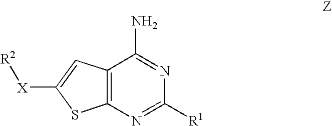

### CROSS-REFERENCE TO RELATED APPLICATIONS

The present application claims the benefits of the filing of U.S. Provisional Application No. 61/104,785 filed Oct. 13, 2008. The complete disclosures of the aforementioned related patent applications are hereby incorporated herein by reference for all purposes.

### FIELD OF THE INVENTION

This invention relates to a novel arylindenopyrimidine and its therapeutic and prophylactic uses. Disorders treated and/or prevented include neurodegenerative and movement disorders ameliorated by antagonizing Adenosine A2a receptors.

### BACKGROUND OF THE INVENTION

Adenosine A2a Receptors Adenosine is a purine nucleotide produced by all metabolically active cells within the body. Adenosine exerts its effects via four subtypes of cell surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology, 1998, 401, 163).

In peripheral tissues, A2a receptors are found in platelets, neutrophils, vascular smooth muscle and endothelium (Gessi, S.; Varani, K.; Merighi, S.; Ongini, E.; Bores, P. A. British Journal of Pharmacology, 2000, 129, 2). The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. Ri; Rivkees, S. A.; Peterfreund, R. A.; Pollack, A. E.; Adler, E. M.; Reppert, S. M. Brain Research Molecular Brain Research, 1992, 14, 186).

Neurochemical studies have shown that activation of A2a receptors reduces the binding affinity of D2 agonist to their receptors. This D2R and A2aR receptor-receptor interaction has been demonstrated instriatal membrane preparations of rats (Ferre, S.; con Euler, G.; Johansson, B.; Fredholm, B. B.; Fuxe, K. Proceedings of the National Academy of Sciences I of the United States of America, 1991, 88, 7238) as well as in fibroblast cell lines after transfected with A2aR and D2R cDNAs (Salim, H.; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP (1-methyl-4-pheny-1,2,3,6-tetrahydropyridine)-induced PC) in various species, including mice, rats, and monkeys (Ikeda, K.; Kurokawa, M.; Aoyana, S.; Kuwana, Y. Journal of Neurochemistry, 2002, 80, 262).

Furthermore, A2a knockout mice with genetic blockade of A2a function have been found to be less sensitive to motor impairment and neurochemical changes when they were exposed to neurotoxin MPTP (Chen, J. F.; Xu, K.; I Petzer, J. P.; Steal, R.; Xu, Y. H.; Beilstein, M.; Sonsalla, P. K.; Castagnoli, K.; Castagnoli, N., Jr.; Schwarsschild, M. A. Journal of Neuroscience, 2001, 121, RC143).

In humans, the adenosine receptor antagonist theophylline has been found to produce beneficial effects in PD patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Hernan, M. A.; Kawachi, I.; Colditz, G. A.; Speizer, F. E.; Willett, W. C. Annals of Neurology, 2001, 50, 56). In summary, adenosine A2a receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist reduces the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31 (8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58 (1), 31-40 and references therein.

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61 (suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28 (2) 197-205.

### SUMMARY OF THE INVENTION

The present invention includes compounds of Formula Z

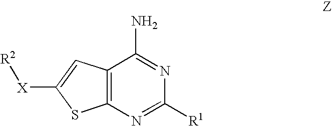

wherein:
X is selected from the group consisting of:

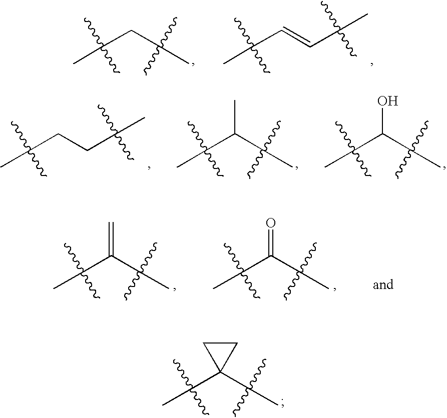

R¹ is heteroaryl optionally substituted with one substituent selected from the group consisting of: —OH, OC₍₁₋₄₎alkyl, CF₃, OCF₃, Cl, Br, —CN, F, CHF₂, C₍₁₋₄₎alkyl, and cyclopropyl;
R² is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN; wherein said C₍₁₋₄₎alkyl is optionally substituted with a ring selected from the group consisting of:

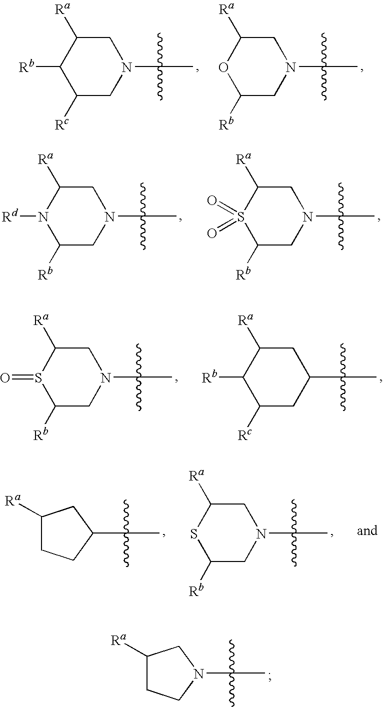

wherein R^{a}, R^{b}, and R^{C} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;
   and solvates, hydrates, tautomers and pharmaceutically acceptable salts thereof.

### DETAILED DESCRIPTION OF THE INVENTION

The present invention includes compounds of Formula Z

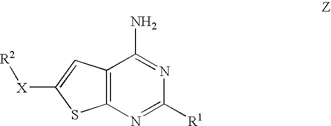

wherein:
X is selected from the group consisting of:

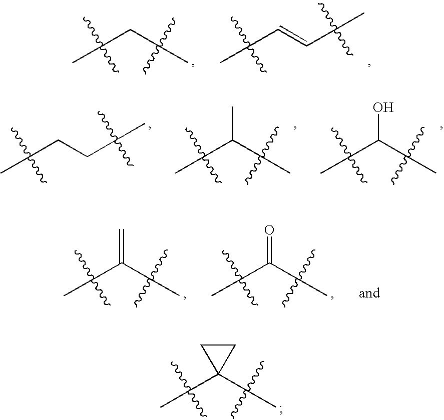

R¹ is heteroaryl optionally substituted with one substituent selected from the group consisting of: —OH, OC₍₁₋₄₎alkyl, CF₃, OCF₃, Cl, Br, —CN, F, CHF₂, C₍₁₋₄₎alkyl, and cyclopropyl;
R² is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN; wherein said C₍₁₋₄₎alkyl is optionally substituted with a ring selected from the group consisting of:

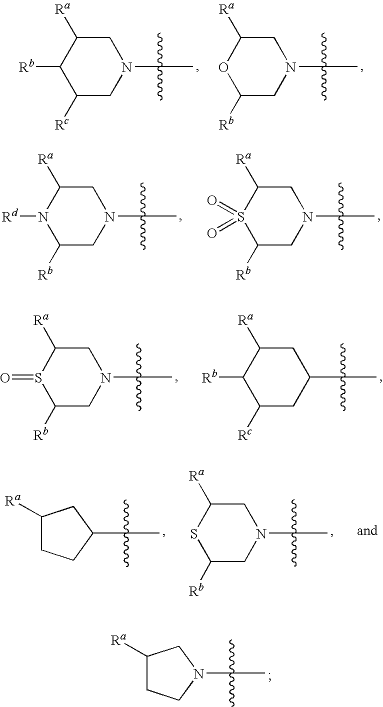

wherein R^{a}, R^{b}, and R^{C} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;
   and solvates, hydrates, tautomers and pharmaceutically acceptable salts thereof.

In another embodiment of the invention:

X is selected from the group consisting of:

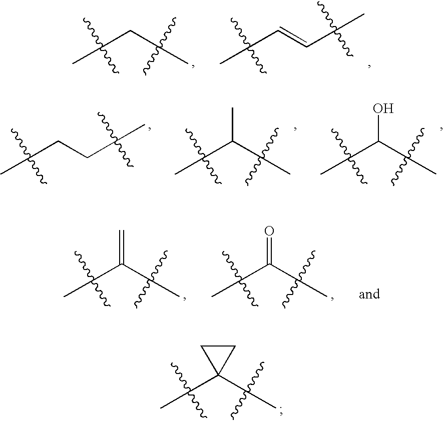

R¹ is heteroaryl optionally substituted with one substituent selected from the group consisting of: —OH, OC₍₁₋₄₎alkyl, OCF₃, Cl, Br, —CN, F, CHF₂, C₍₁₋₄₎alkyl, and cyclopropyl;
R² is phenyl substituted with one substituent, selected from the group consisting of: H, —OH, OC₍₁₋₄₎alkyl, OCF₃, CHF₂, CF₃, Cl, Br, —CN, F, and C₍₁₋₄₎alkyl, wherein said C₍₁₋₄₎alkyl is optionally substituted with morpholinyl, piperidinyl, or piperazinyl;
and solvates, hydrates, tautomers and pharmaceutically acceptable salts thereof.

In another embodiment of the invention:

X is selected from the group consisting of:

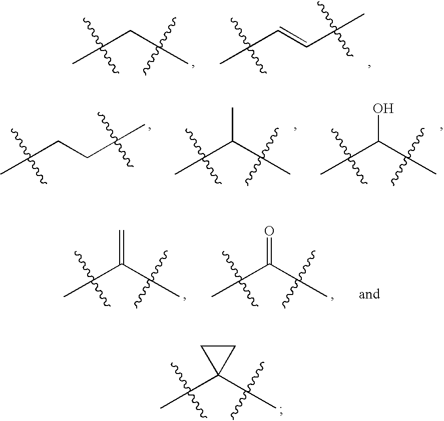

R¹ is heteroaryl selected from the group consisting of: furyl, thiazolyl, pyridyl, oxazolyl, imidazolyl, pyrimidyl, thiophenyl, and pyridazyl, wherein said heteroaryl is optionally substituted with one substituent selected from the group consisting of: OC₍₁₋₄₎alkyl, OCF₃, —CN, F, CHF₂, C₍₁₋₄₎alkyl, and cyclopropyl;
R² is phenyl substituted with one substituent, selected from the group consisting of: H, —OH, OC₍₁₋₄₎alkyl, OCF₃, CHF₂, CF₃, F, Cl, and C₍₁₋₄₎alkyl, wherein said C₍₁₋₄₎alkyl is optionally substituted with morpholinyl, piperidinyl, or piperazinyl;
and solvates, hydrates, tautomers and pharmaceutically acceptable salts thereof.

In another embodiment of the invention:

X is selected from the group consisting of:

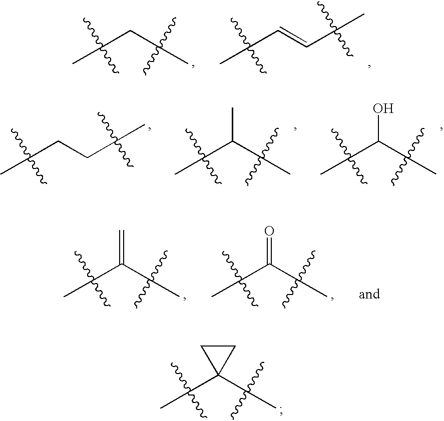

R¹ is heteroaryl selected from the group consisting of: furyl, thiazolyl, pyridyl, oxazolyl, imidazolyl, pyrimidyl, thiophenyl, and pyridazyl, wherein said heteroaryl is optionally substituted with one substituent selected from the group consisting of: C₍₁₋₄₎alkyl, OCH₃, —CN, CHF₂, and cyclopropyl;
R² is phenyl substituted with one substituent, selected from the group consisting of: H, —OH, OCH₃, OCF₃, CHF₂, CF₃, F, Cl, and C₍₁₋₄₎alkyl, wherein said C₍₁₋₄₎alkyl is optionally substituted with morpholinyl;
and solvates, hydrates, tautomers and pharmaceutically acceptable salts thereof.

In another embodiment of the invention:

X is selected from the group consisting of:

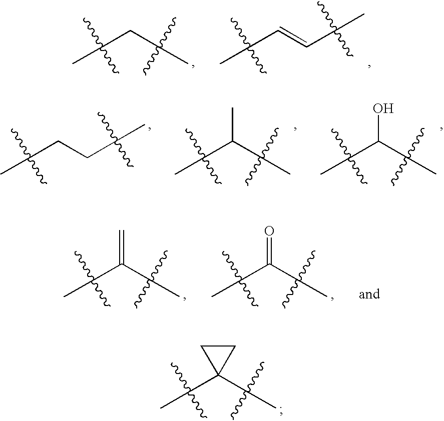

R¹ is selected from the group consisting of:

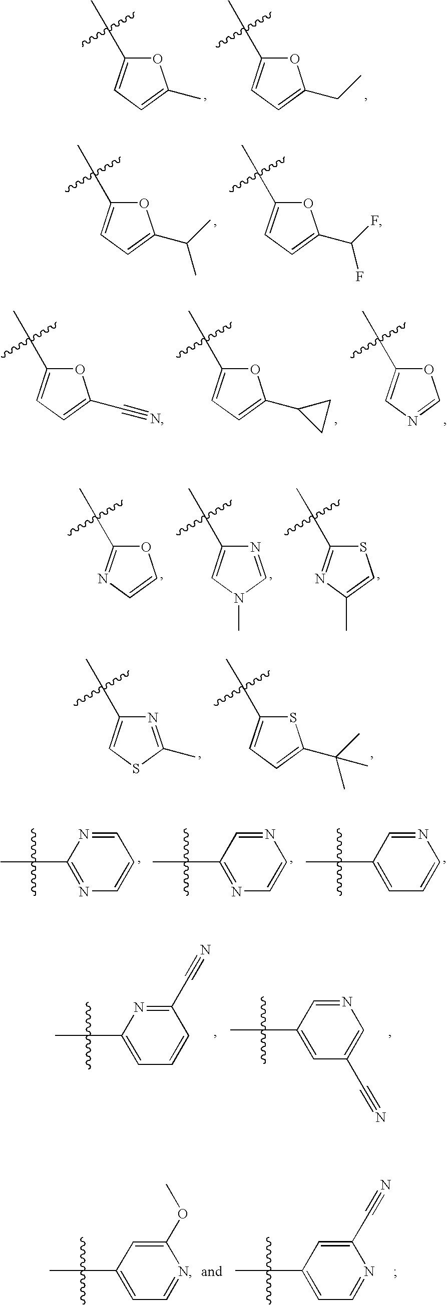

R² is phenyl substituted with one substituent, selected from the group consisting of: H, —CH₂-morpholinyl, OCH₃, F, and Cl;
and solvates, hydrates, tautomers, and pharmaceutically acceptable salts thereof.

Another embodiment of the invention comprises a compound selected from the group consisting of:

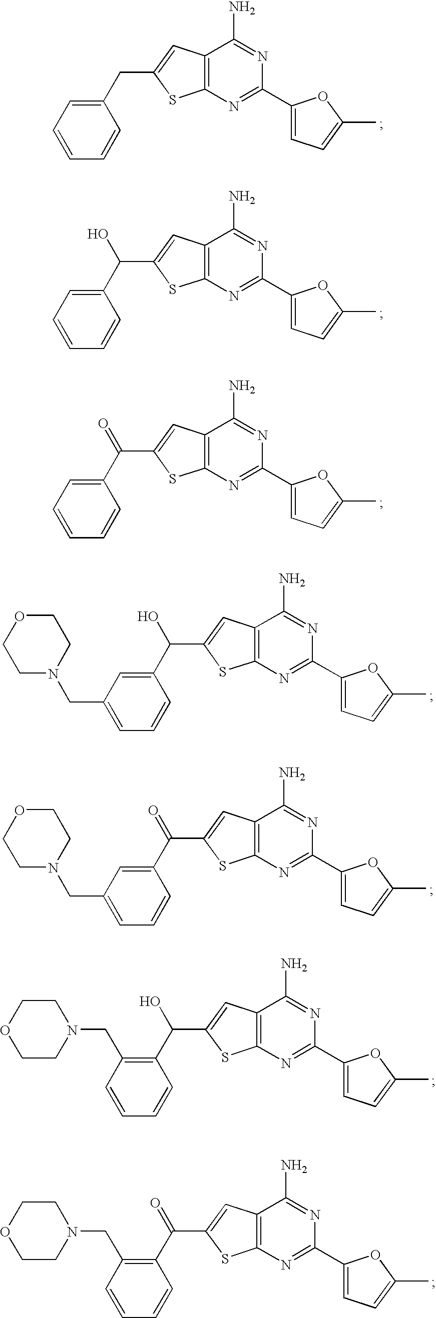

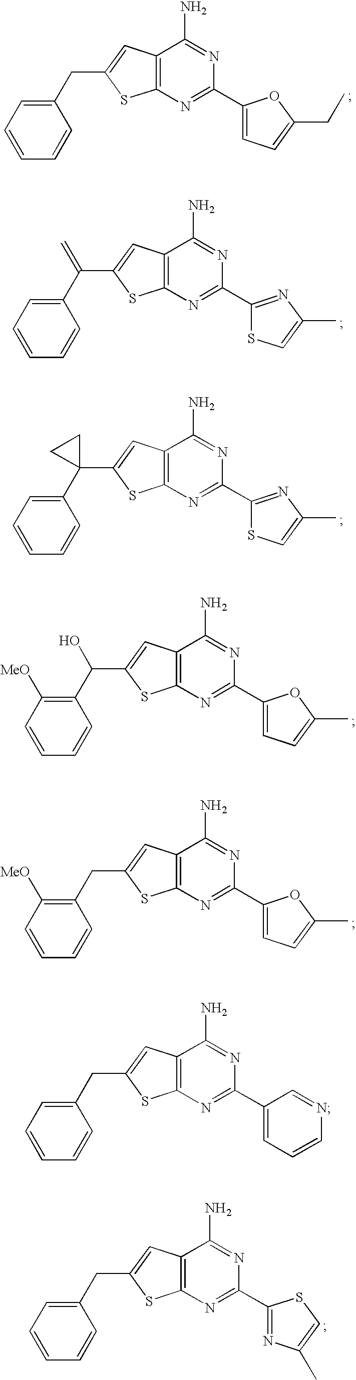

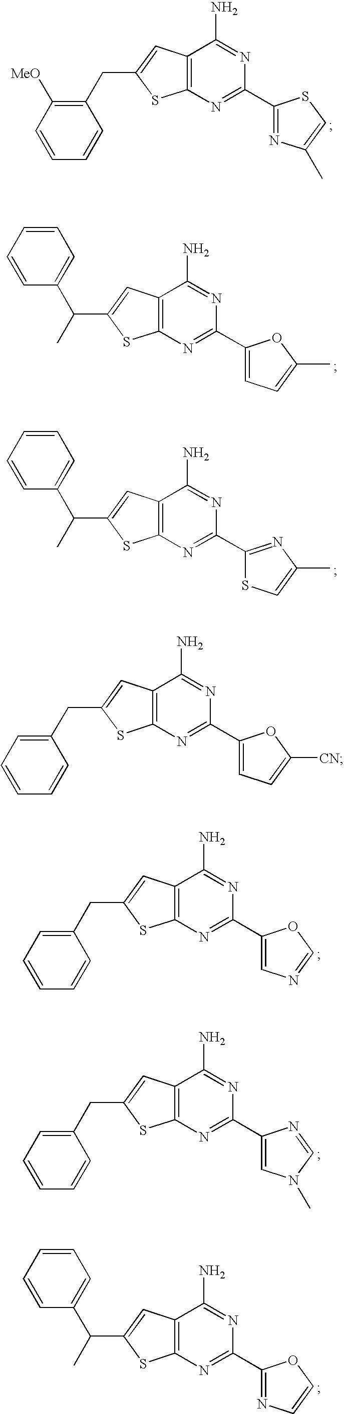

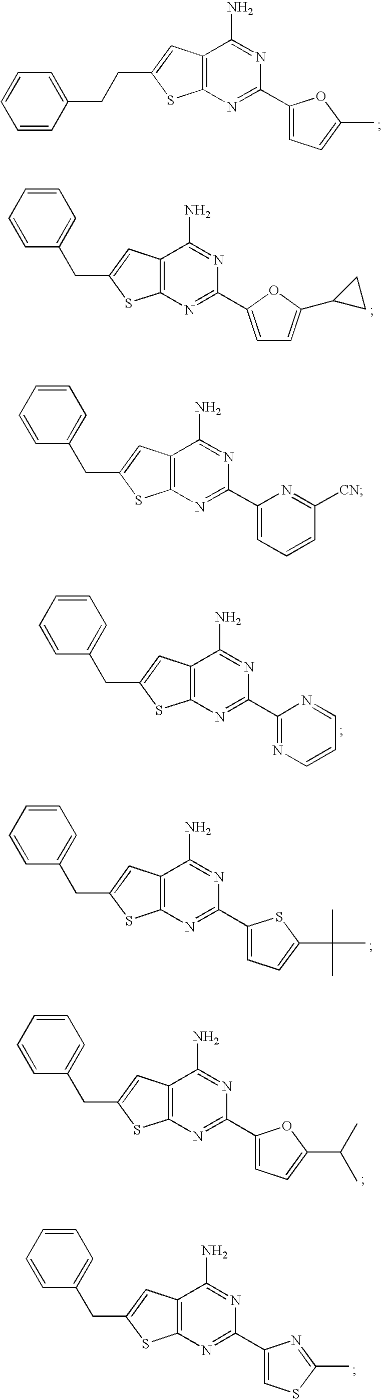

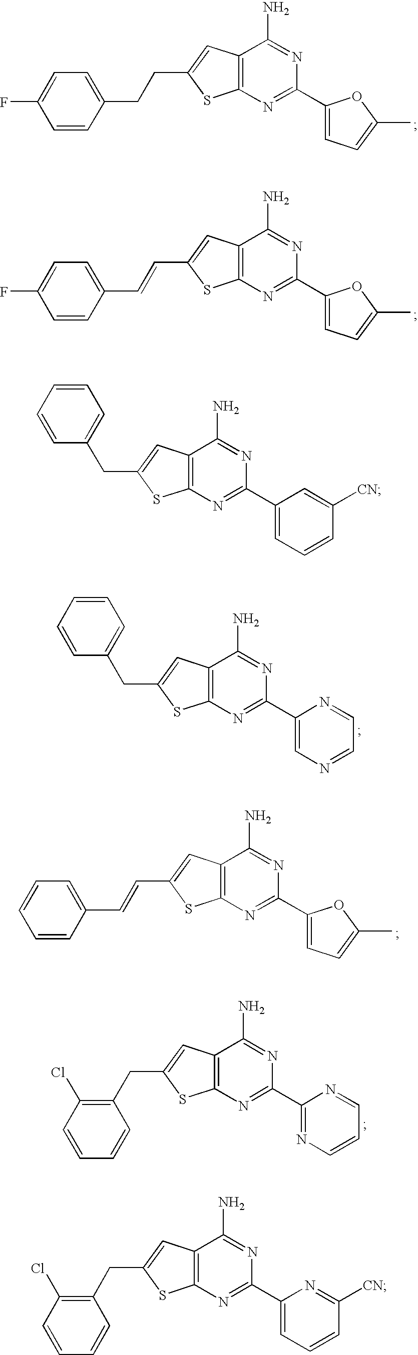

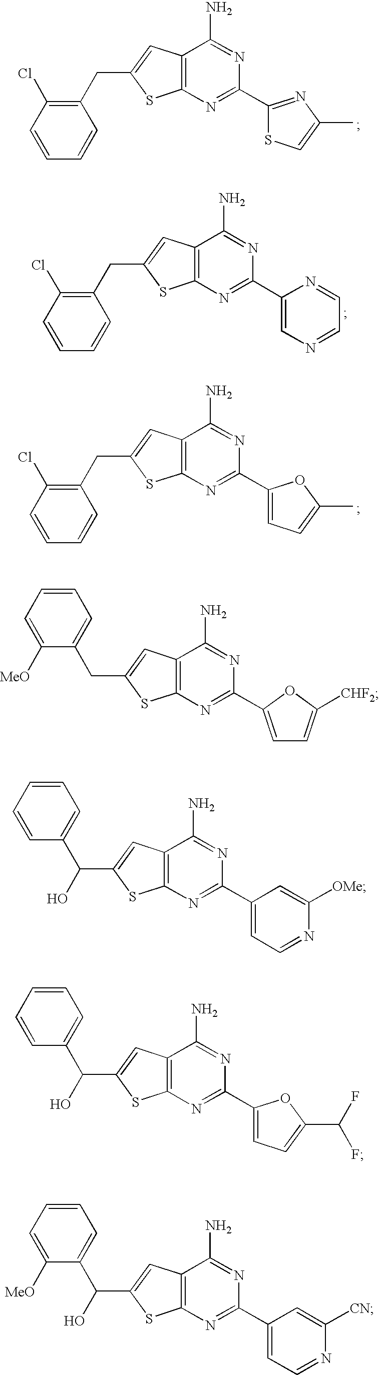

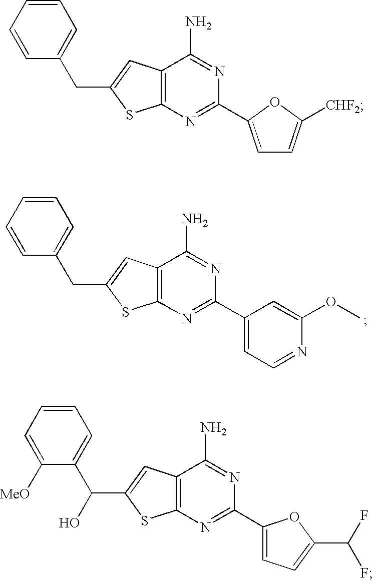

and solvates, hydrates, tautomers and pharmaceutically acceptable salts thereof.

This invention further provides a method of treating a subject having a condition ameliorated by antagonizing Adenosine A2a receptors, which comprises administering to the subject a therapeutically effective dose of a compound of Formula Z.

This invention further provides a method of preventing a disorder ameliorated by antagonizing Adenosine A2a receptors in a subject, comprising of administering to the subject a prophylactically effective dose of the compound of claim 1 either preceding or subsequent to an event anticipated to cause a disorder ameliorated by antagonizing Adenosine A2a receptors in the subject.

Compounds of Formula Z can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharic.

This invention also provides a pharmaceutical composition comprising a compound of Formula Z and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A2a receptors, which comprises administering to the subject a therapeutically effective dose of a compound of Formula Z.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A2a receptors. In a preferred embodiment, the subject is a human.

Administering the instant pharmaceutical composition can be effected or performed using any of the various methods known to those skilled in the art. Compounds of Formula Z can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of Formula Z. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0, ug/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of Formula Z.

The invention also provides a method of treating ADHD in a mammal, comprising administering a therapeutically effective dose of a compound of Formula Z.

The invention also provides a method of treating depression in a mammal, comprising administering a therapeutically effective dose of a compound of Formula Z.

The invention also provides a method of treating anxiety in a mammal, comprising administering a therapeutically effective dose of a compound of Formula Z.

### DEFINITIONS

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “cycloalkyl” refers to a radical derived by the removal of one hydrogen atom from a ring carbon atom of a saturated alkyl ring system. Typical cycloalkyl radicals include cyclopropyl, cyclobutyl, cyclopentyl, cyclohexyl, cycloheptyl, and cyclooctyl.

The term “heteroaryl” refers to a radical derived by the removal of one hydrogen atom from a ring carbon atom of a heteroaromatic ring system. Typical heteroaryl radicals include furyl, pyrrolyl, oxazolyl, thiophenyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, indazolyl, benzimidazolyl, benzothiazolyl, purinyl, 4H-quinolizinyl, quinolinyl, isoquinolinyl, cinnolinyl, phthalzinyl, quinazolinyl, quinoxalinyl, 1,8-naphthyridinyl, and pteridinyl.

The term “heterocyclyl” refers to a radical derived by the removal of one hydrogen atom from a ring carbon or ring nitrogen atom of a saturated or partially saturated heteroaromatic ring system. Typical heterocyclyl radicals include morpholinyl, piperidinyl, piperazinyl, pyrrolidinyl, and tetrahydrofuranyl.

### ABBREVIATIONS

Herein and throughout this application, the following abbreviations may be used.

BOC butyloxycarbonyl
n-BuLi n-butyllithium
t-BuOK potassium tert-butoxide
Cy cyclopropyl
DMF dimethylformamide
DMAP dimethylaminopyridine
DMSO dimethylsulfoxide
Et ethyl
LDA lithium diisopropylamine
Me methyl
NBS N-bromo succinimide
OAc acetate
Pd(dppf)Cl_{2 [}1,1′-Bis(diphenylphosphino)ferrocene]dichloropalladium (II)
py pyridine
TFA trifluoroacetic acid
THF tetrahydrofuran

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, Ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution.

The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

### General Schemes:

Compounds of Formula Z can be prepared by methods known to those who are skilled in the art. The following reaction schemes are only meant to represent examples of the invention and are in no way meant to be a limit of the invention.

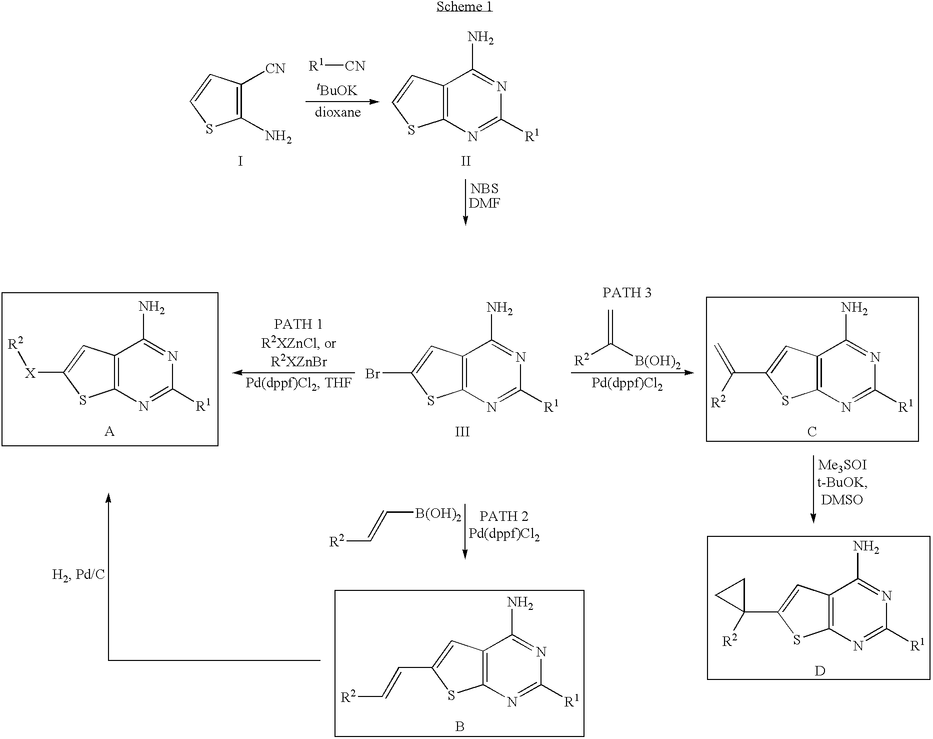

Scheme 1 illustrates the synthetic routes (Paths 1, 2 and 3) leading to compounds of Formula Z (A, B, C, and D). Starting with 2-amino-3-cyanothiophene I and following the path indicated by the arrows, condensation under basic conditions with R¹—CN, where R¹ is as defined in Formula Z, affords the aminopyrimidine II. The aminopyrimidine II is reacted with N-bromosuccinimide (NBS), to give the bromothiophene III. Following path 1 bromothiophene III is reacted with R²XZnCl or R²XZnBr, where R² is as defined in Formula Z, in the presence of a palladium catalyst to afford compounds of Formula Z, where X is CH₂ or CH₂CH₂ (A). Alternatively, compounds of Formula B can be reduced by hydrogenation to give compounds of Formula Z, where X is

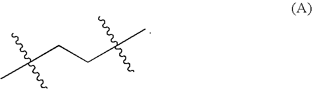

Following path 2 bromothiophene III is reacted with R²CHCHB(OH)₂, where R² is as defined in Formula Z, in the presence of a palladium catalyst to give compounds of Formula Z, where X is

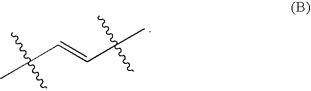

Following path 3 bromothiophene III is reacted with R²C(CH₂)B(OH)₂, where R² is as defined in Formula Z, in the presence of palladium to give compounds of Formula Z, where X is

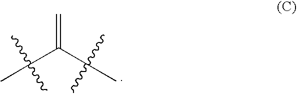

Compounds of Formula C are reacted with trimethylsufoxonium iodide under basic conditions to afford compounds of Formula Z, where X is

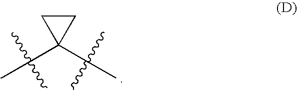

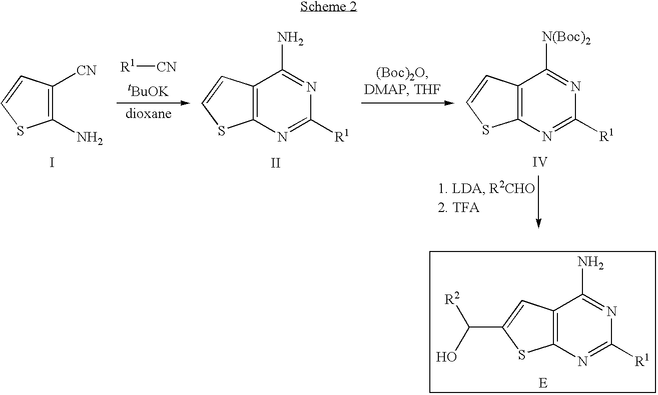

Starting with 2-amino-3cyanothiophene I and following the path indicated by the arrows, condensation under basic conditions with R¹—CN, where R¹ is as defined in Formula Z, affords the aminopyrimidine II. The aminopyrimidine II is reacted with di-tert-butyldicarbonate [(Boc)₂O] in the presence of 4-dimethylamino pyridine (DMAP) to give the corresponding protected amine IV. The thiophene IV is deprotonated with lithium diisopropylamide (LDA) and reacted with R²CHO, where R² is as defined in Formula Z, to give an intermediate alcohol that is deprotected with TFA to give compounds of Formula E.

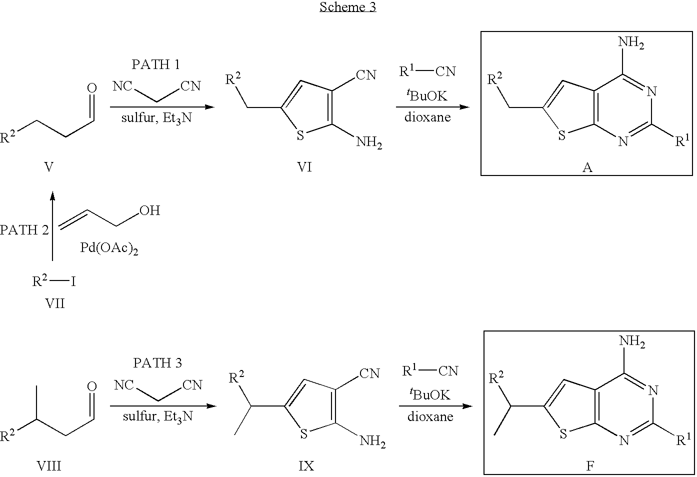

Scheme 3 illustrates an alternative method of synthesizing compounds of Formula A, as well as a method of accessing compounds of Formula Z where X is

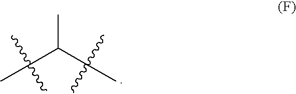

Starting with R²CH₂CH₂CHO (V), where R² is as defined in Formula A, reaction with malononitrile and elemental sulfur under basic conditions gives the thiophene VI. The thiophene VI is condensed under basic conditions with R¹—CN, where R¹ is as defined in Formula Z, to afford compounds of Formula Z where X is CH₂ (A). Alternatively aldehydes that are not commercially available can be synthesized following path 2 where R²—I (VII), where R² is as defined in Formula Z, is reacted with allyl alcohol in the presence of a palladium catalyst to give aldehydes V that then follow the arrows in path 1. Following path 3, methyl substituted aldehydes VIII react in a similar way to aldehydes in path 1 to afford the methyl substituted compounds of the Formula F.

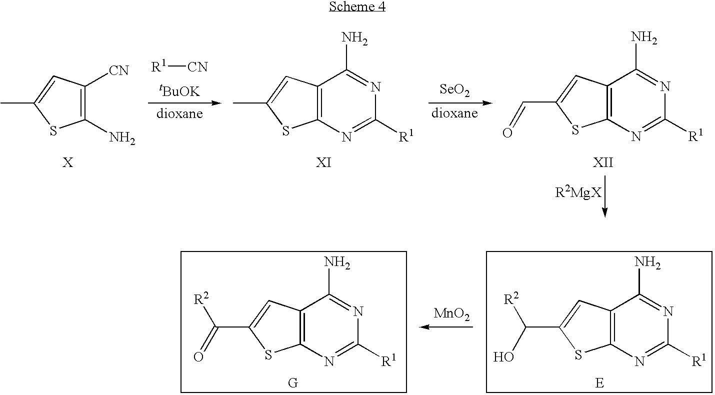

Scheme 4 illustrates the synthetic route leading to compounds of Formulae E and G. Starting with 2-amino-5-methyl-thiophene-3-carbonitrile X condensation under basic conditions with R¹—CN, where R¹ is as defined in Formula Z, affords the aminopyrimidine XI. Oxidation of XI with SeO₂ affords the corresponding aldehyde XII. The aldehyde XII is reacted with R²MgX, where R² is as defined in Formula Z, to give the compounds of the Formula E, which may be oxidized to the corresponding ketone.

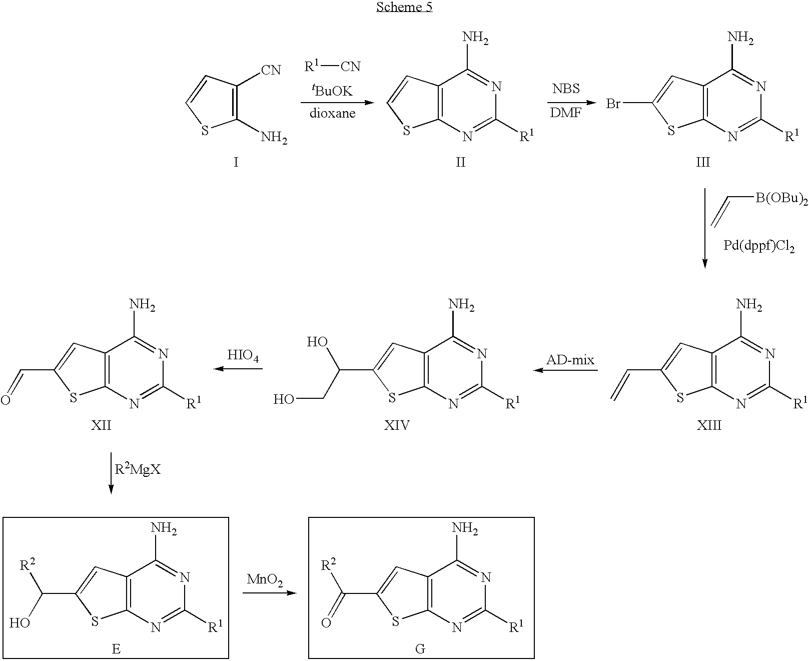

Scheme 5 illustrates an alternative synthetic route to compounds of Formulae E and G. Starting with 2-amino-3-cyanothiophene I and following the path indicated by the arrows, condensation under basic conditions with R¹—CN, where R¹ is as defined in Formula Z, affords the aminopyrimidine II. The aminopyrimidine II is reacted with N-bromosuccinimide (NBS), to give the bromothiophene III. Palladium catalyzed coupling with vinylboronic acid dibutyl ester affords the corresponding vinyl adduct XIII. The olefin present in XIII can be dihydroxylated using AD-mix to give diol XIV that is then oxidized using periodic acid to afford the aldehyde XII. The aldehyde XII is reacted with R²MgX, where R² is as defined in Formula Z, to give the compounds of the Formula E that are oxidized to the corresponding ketone to give compounds of the Formula G.

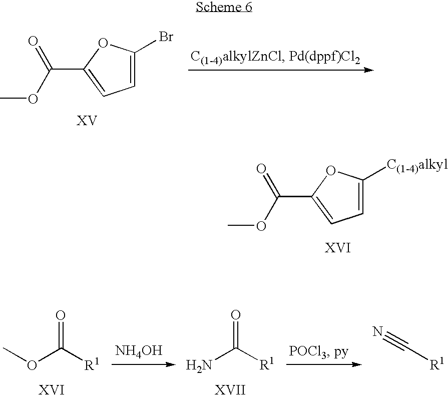

Scheme 6 illustrates the synthetic route to compounds of Formula R¹—CN, where R¹ is a C₍₁₋₄₎alkyl substituted furan. Scheme 6 also illustrates how any R¹—CO₂CH₃ may be converted into R¹—CN. Bromofuran XV can react with alkylzinc reagents in the presence of a palladium catalyst to give XVI. Ester XVI (or any R¹—CO₂CH₃) is reacted with ammonium hydroxide to give the corresponding amide XVII. Dehydration of the amide is accomplished using POCl₃ in pyridine to give the desired heterocyclic nitrile R¹—CN.

### EXAMPLES

### Example 1

### 6-Benzyl-2-(5-methyl-furan-2-yl)-thieno[2,3-d]pyrimidin-4-ylamine

### Example 1

### Step a

### 2-(5-Methyl-furan-2-yl)-thieno[2,3-d]pyrimidin-4-ylamine

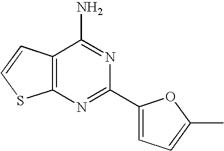

Solid t-BuOK (904 mg, 8.1 mmol) was added to a dioxane suspension (20 mL) of 2-amino-thiophene-3-carbonitrile (5.0 g, 40.3 mmol) and 5-methyl-furan-2-carbonitrile (4.5 g, 40.3 mmol) and the mixture was immersed into a 130° C. oil bath. After 10 min the flask was removed from the oil bath, diluted with THF, filtered and dry packed onto silica gel. Column chromatography gave 5.8 g of 2-(5-methyl-furan-2-yl)-thieno[2,3-d]pyrimidin-4-ylamine.

### Example 1

### Step b

### 6-Bromo-2-(5-methyl-furan-2-yl)-thieno[2,3-d]pyrimidin-4-ylamine

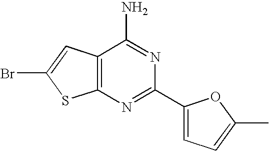

Solid NBS (4.7 g, 26.4 mmol) was added to a THF solution (100 mL) of 2-(5-methyl-furan-2-yl)-thieno[2,3-d]pyrimidin-4-ylamine (5.8 g, 25.1 mmol). After 2 h the mixture was diluted with EtOAc and washed consecutively with saturated aqueous NaHCO₃, 1 M aqueous Na₂S₂O₃, and brine. The organic layer was dried (Na₂SO₄) and dry packed onto silica gel. Column chromatography gave 6.3 g of 6-bromo-2-(5-methyl-furan-2-yl)-thieno[2,3-d]pyrimidin-4-ylamine.

### Example 1

### Step c

### 6-Benzyl-2-(5-methyl-furan-2-yl)-thieno[2,3-d]pyrimidin-4-ylamine

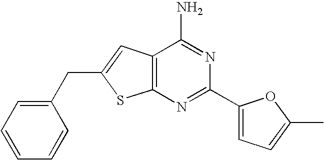

A 0.5 M THF solution of benzylzinc bromide (30 mL, 15 mmol) was added to a THF solution (30 mL) of 6-bromo-2-(5-methyl-furan-2-yl)-thieno[2,3-d]pyrimidin-4-ylamine (1.3 g, 4.3 mmol) and Pd(dppf)Cl₂ (351 mg, 0.4 mmol) and the mixture was heated to reflux. After 6 h the mixture was diluted with EtOAc, washed with water then brine, dried (Na₂SO₄), and dry packed onto silica gel. Column chromatography gave 912 mg of 6-Benzyl-2-(5-methyl-furan-2-yl)-thieno[2,3-d]pyrimidin-4-ylamine. ¹H NMR (Acetone, 400 MHz): δ=7.31-7.37 (m, 4H), 7.22-7.31 (m, 1H), 7.20 (d, J=1.3 Hz, 1H), 7.02 (d, J=3.0 Hz, 1H), 6.72 (br. s, 2H), 6.15-6.20 (m, 1H), 4.23 (s, 2H), 2.36 ppm (s, 3H); MS m/e 322 (M+H).

### Example 2

### [4-Amino-2-(5-methyl-furan-2-yl)-thieno[2,3-d]pyrimidin-6-yl]-phenyl-methanol

### Example 2

### Step a

### 2-(5-Methyl-furan-2-yl)-6-vinyl-thieno[2,3-d]pyrimidin-4-ylamine

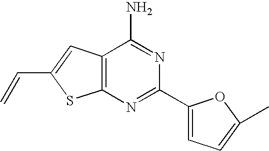

Neat vinylboronic acid dibutyl ester (1.2 mL, 5.3 mmol) was added to a dioxane (24 mL)/water (6 mL) solution of 6-bromo-2-(5-methyl-furan-2-yl)-thieno[2,3-d]pyrimidin-4-ylamine (825 mg, 2.7 mmol, an intermediate from Example 1), Pd(dppf)Cl₂ (217 mg, 0.3 mmol), and K₂CO₃ (735 mg, 5.3 mmol) and the mixture was heated to 80° C. After 5 h the mixture was cooled and diluted with EtOAc. The organic phase was washed with water then brine, dried (Na₂SO₄) and dry packed onto silica gel. Column chromatography gave 550 mg of the title compound.

### Example 2

### Step b

### 1-[4-Amino-2-(5-methyl-furan-2-yl)-thieno[2,3-d]pyrimidin-6-yl]-ethane-1,2-diol

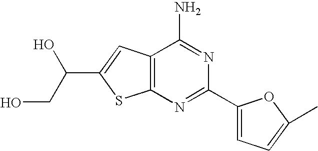

Solid MeSO₂NH₂ (204 mg, 2.1 mmol) was added to a t-BuOH (10 mL)/water (10 mL) solution of AD mix-α (3.0 g). After 15 min the resulting mixture was added to solid 2-(5-methyl-furan-2-yl)-6-vinyl-thieno[2,3-d]pyrimidin-4-ylamine (550 mg, 2.1 mmol) and the mixture was stirred vigorously. After 17 h solid sodium sulfite (3.7 g) was added and the mixture was stirred for an additional 30 minutes. The mixture was extracted with EtOAc and the combined extracts were washed with water and brine, dried (Na₂SO₄), concentrated, and purified via column chromatography to give 565 mg of the title compound.

### Example 2

### Step c

### 4-Amino-2-(5-methyl-furan-2-yl)-thieno[2,3-d]pyrimidine-6-carbaldehyde

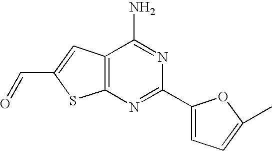

Solid HIO₄ (877 mg, 3.9 mmol) was added to a THF solution (20 mL) of 1-[4-amino-2-(5-methyl-furan-2-yl)-thieno[2,3-d]pyrimidin-6-yl]-ethane-1,2-diol (560 mg, 1.9 mmol). After 1 h saturated aqueous NaHCO₃ was added and the aqueous phase was extracted with EtOAc. The combined extracts were washed with water and brine, dried (Na₂SO₄), concentrated, and purified via column chromatography to give 460 mg of the title compound.

### Example 2

### Step d

### [4-Amino-2-(5-methyl-furan-2-yl)-thieno[2,3-d]pyrimidin-6-yl]-phenyl-methanol

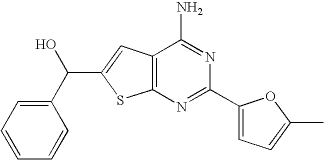

A 1.0 M THF solution of phenylmagnesium bromide (0.97 ml, 0.97 mmol) was added to a 0° C. THF solution (4 mL) of 4-amino-2-(5-methyl-furan-2-yl)-thieno[2,3-d]pyrimidine-6-carbaldehyde (100 mg, 0.39 mmol). After 15 min water was added and the mixture was extracted with EtOAc. The combined extracts were washed with water and brine, dried (Na₂SO₄), concentrated, and purified via column chromatography to give 81 mg of the title compound. ¹H NMR (CHLOROFORM-d, 400 MHz): δ=7.43-7.47 (m, 2H), 7.30-7.41 (m, 5H), 7.15 (d, J=3.3 Hz, 1H), 6.78 (d, J=110 Hz, 1H), 6.13 (d, J=3.3 Hz, 1H), 6.01 (s, 1H), 5.28 (s, 2H), 2.42 (s, 3H); MS m/e 338 (M+H).

### Example 3

### [4-Amino-2-(5-methyl-furan-2-yl)-thieno[2,3-d]pyrimidin-6-yl]-phenyl-methanone

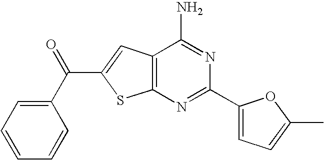

Solid MnO₂ (286 mg, 3.30 mmol) was added to a CH₂Cl₂ solution (2 mL) of [4-amino-2-(5-methyl-furan-2-yl)-thieno[2,3-d]pyrimidin-6-yl]-phenyl-methanol (37 mg, 0.11 mmol, prepared as described in Example 2) and the suspension was stirred vigorously. After 18 h the mixture was filtered through Celite and the filtrate was concentrated to give 33 mg of the title compound that analytically pure. ¹H NMR (DMSO-d₆, 400 MHz): δ=8.34 (s, 1H), 8.15 (br. s, 2H), 7.88 (d, J=7.1 Hz, 2H), 7.73 (t, J=7.3 Hz, 1H), 7.63 (t, J=7.7 Hz, 2H), 7.22 (d, J=3.0 Hz, 1H), 6.34 (d, J=3.3 Hz, 1H), 2.40 ppm (s, 3H); MS m/e 336 (M+H).

### Example 4

### [4-Amino-2-(5-methyl-furan-2-yl)-thieno[2,3-d]pyrimidin-6-yl]-(3-morpholin-4-ylmethyl-phenyl)-methanol

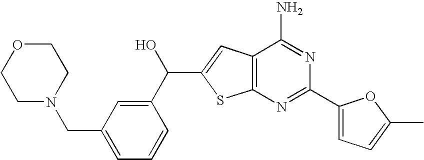

The title compound was prepared using (3-(4-morpholinylmethyl)phenyl)magnesium bromide in place of phenylmagnesium bromide as described in Example 2. ¹H NMR (CHLOROFORM-d, 400 MHz): δ=7.44 (s, 1H), 7.28-7.37 (m, 3H), 7.15 (d, J=3.3 Hz, 1H), 6.84 (d, J=1.0 Hz, 1H), 6.10-6.17 (m, 1H), 6.01 (s, 1H), 5.28 (s, 2H), 3.64-3.70 (m, 4H), 3.49 (s, 2H), 2.38-2.45 ppm (m, 7H); MS m/e 437 (M+H).

### Example 5

### [4-Amino-2-(5-methyl-furan-2-yl)-thieno[2,3-d]pyrimidin-6-yl]-(3-morpholin-4-ylmethyl-phenyl)-methanone

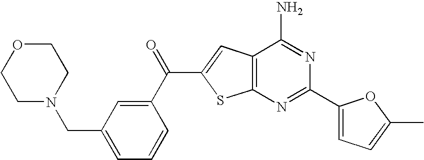

The title compound was prepared using [4-amino-2-(5-methyl-furan-2-yl)-thieno[2,3-d]pyrimidin-6-yl]-(3-morpholin-4-ylmethyl-phenyl)-methanol (prepared as described in Example 4) in place of [4-amino-2-(5-methyl-furan-2-yl)-thieno[2,3-d]pyrimidin-6-yl]-phenyl-methanol as described in Example 3. ¹H NMR (CHLOROFORM-d, 400 MHz): δ=7.85 (s, 1H), 7.77 (d, J=7.8 Hz, 1H), 7.74 (s, 1H), 7.60 (d, J=7.6 Hz, 1H), 7.48 (t, J=7.7 Hz, 1H), 7.29 (d, J=3.3 Hz, 1H), 6.20 (d, J=3.3 Hz, 1H), 5.58 (br. s, 2H), 3.69-3.76 (m, 4H), 3.59 (s, 2H), 2.49 (t, J=4.3 Hz, 4H), 2.47 ppm (s, 3H); MS m/e 435 (M+H).

### Example 6

### [4-Amino-2-(5-methyl-furan-2-yl)-thieno[2,3-d]pyrimidin-6-yl]-(2-morpholin-4-ylmethyl-phenyl)-methanol

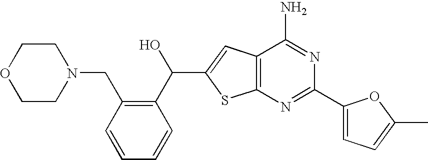

The title compound was prepared using 2-[(4-morpholino)methyl]phenylmagnesium bromide in place of phenylmagnesium bromide as described in Example 2. ¹H NMR (CHLOROFORM-d, 400 MHz): δ=7.37-7.45 (m, 2H), 7.30-7.37 (m, 1H), 7.24 (d, J=7.3 Hz, 1H), 7.16 (d, J=3.3 Hz, 1H), 6.61 (d, J=1.8 Hz, 1H), 6.14 (dd, J=3.3, 1.0 Hz, 1H), 5.94 (s, 1H), 5.18 (s, 2H), 3.67-3.77 (m, 4H), 3.65 (d, J=12.4 Hz, 2H), 3.17 (d, J=12.6 Hz, 2H), 2.43-2.50 ppm (m, 7H); MS m/e 437 (M+H).

### Example 7

### [4-Amino-2-(5-methyl-furan-2-yl)-thieno[2,3-d]pyrimidin-6-yl]-(2-morpholin-4-ylmethyl-phenyl)-methanone

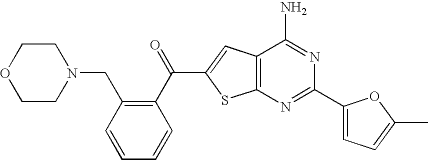

The title compound was prepared using [4-amino-2-(5-methyl-furan-2-yl)-thieno[2,3-d]pyrimidin-6-yl]-(2-morpholin-4-ylmethyl-phenyl)-methanol (prepared as described in Example 6) in place of [4-amino-2-(5-methyl-furan-2-yl)-thieno[2,3-d]pyrimidin-6-yl]-phenyl-methanol as described in Example 3. ¹H NMR (CHLOROFORM-d, 300 MHz): δ=7.31-7.48 (m, 4H), 7.28 (d, J=3.4 Hz, 1H), 7.22 (br. s, 1H), 6.20 (dd, J=3.3, 0.8 Hz, 1H), 5.55 (br. s, 2H), 3.46 (br. s, 2H), 2.85-3.30 (m, 4H), 2.46 (s, 3H), 1.80-2.30 (m, 4H); MS m/e 435 (M+H).

### Example 8

### 6-Benzyl-2-(5-ethyl-furan-2-yl)-thieno[2,3-d]pyrimidin-4-ylamine

### Example 8

### Step a

### 5-Ethyl-furan-2-carboxylic acid methyl ester

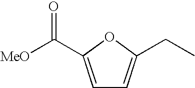

A 1.0 M heptane solution of Et₂Zn (73 mL, 73.0 mmol) was added to a THF solution (100 mL) of 5-bromo-furan-2-carboxylic acid methyl ester (5.0 g, 24.4 mmol) and Pd(dppf)Cl₂ (2.0 g, 2.4 mmol) and the mixture was heated to reflux. After 16 h the mixture was cooled to rt and water was added dropwise to quench the excess Et₂Zn. The mixture was extracted with EtOAc and the combined organics were washed with water then brine, dried (Na₂SO₄) and concentrated to give 2.8 g of 5-ethyl-furan-2-carboxylic acid methyl ester that was used without further purification.

### Example 8

### Step b

### 5-Ethyl-furan-2-carboxylic acid amide

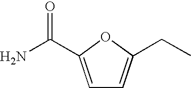

5-Ethyl-furan-2-carboxylic acid methyl ester (2.8 g, 18.2 mmol) was suspended in concentrated NH₄OH (80 mL) and stirred vigorously. After 24 h the white slurry was diluted with water, filtered, and the collected solid was washed with water and dried in vacuo to give 1.5 g of 5-ethyl-furan-2-carboxylic acid amide.

### Example 8

### Step c

### 5-Ethyl-furan-2-carbonitrile

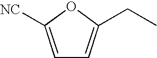

Neat POCl₃ (0.56 mL, 6.0 mmol) was added to a pyridine solution (11 mL) of 5-ethyl-furan-2-carboxylic acid amide (600 mg, 4.3 mmol). After 2 h the mixture was cooled to 0° C., diluted with water, and adjusted to pH 4.5 with concentrated HCl. The dark mixture was extracted with Et₂O, dried (Na₂SO₄), and concentrated to give 520 mg of 5-ethyl-furan-2-carbonitrile that was used immediately without further purification.

### Example 8

### Step d

### 2-(5-Ethyl-furan-2-yl)-thieno[2,3-d]pyrimidin-4-ylamine

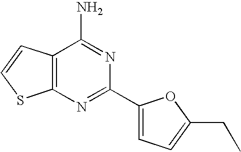

Solid t-BuOK (121 mg, 1.1 mmol) was added to a dioxane solution (1 mL) of 5-ethyl-furan-2-carbonitrile (520 mg, 4.3 mmol) and 2-amino-thiophene-3-carbonitrile (533 mg, 4.3 mmol) and the mixture was immersed into a 130° C. oil bath. After 15 min the mixture was removed from the oil bath, diluted with THF, and dry packed onto silica gel. Column chromatography gave 490 mg of 2-(5-ethyl-furan-2-yl)-thieno[2,3-d]pyrimidin-4-ylamine.

### Example 8

### Step e

### 6-Bromo-2-(5-ethyl-furan-2-yl)-thieno[2,3-d]pyrimidin-4-ylamine

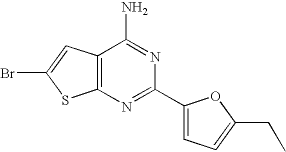

Solid NBS (330 mg, 1.9 mmol) was added to a THF solution (15 mL) of 2-(5-ethyl-furan-2-yl)-thieno[2,3-d]pyrimidin-4-ylamine (433 mg, 1.8 mmol). After 3 h the mixture was diluted with EtOAc, washed with water then brine, dried (Na₂SO₄), and dry packed onto silica gel. Column chromatography gave 464 mg of 6-bromo-2-(5-ethyl-furan-2-yl)-thieno[2,3-d]pyrimidin-4-ylamine.

### Example 8

### Step f

### 6-Benzyl-2-(5-ethyl-furan-2-yl)-thieno[2,3-d]pyrimidin-4-ylamine

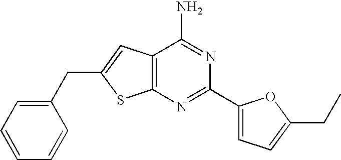

A 0.5 M THF solution of BnZnBr (0.91 mL, 0.46 mmol) was added to a THF solution of 6-bromo-2-(5-ethyl-furan-2-yl)-thieno[2,3-d]pyrimidin-4-ylamine (37 mg, 0.11 mmol) and Pd(dppf)Cl₂ (9 mg, 0.01 mmol) and the mixture was heated to reflux. After 3 h the mixture was diluted with EtOAc, washed with water then brine, dried (Na₂SO₄), and dry packed onto silica gel. Column chromatography gave 22 mg of 6-benzyl-2-(5-ethyl-furan-2-yl)-thieno[2,3-d]pyrimidin-4-ylamine. ¹H NMR (CHLOROFORM-d, 300 MHz): δ=7.32-7.39 (m, 2H), 7.27-7.31 (m, 3H), 7.16 (d, J=3.0 Hz, 1H), 6.68 (s, 1H), 6.15 (d, J=3.4 Hz, 1H), 5.17 (br. s, 2H), 4.18 (s, 2H), 2.81 (q, J=7.4 Hz, 2H), 1.29 ppm (t, J=7.5 Hz, 3H); MS m/e 336 (M+H).

### Example 9

### 2-(4-Methyl-thiazol-2-yl)-6-(1-phenyl-vinyl)-thieno[2,3-d]pyrimidin-4-ylamine

### Example 9

### Step a

### 2-(4-Methyl-thiazol-2-yl)-thieno[2,3-d]pyrimidin-4-ylamine

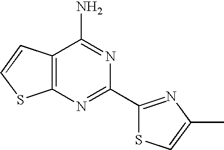

The title compound was prepared using 4-methylthiazole-2-carbonitrile in place of and 5-methyl-furan-2-carbonitrile as described in Example 1.

### Example 9

### Step b

### 6-Bromo-2-(4-methyl-thiazol-2-yl)-thieno[2,3-d]pyrimidin-4-ylamine

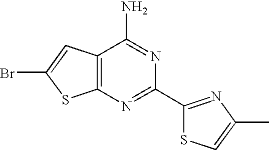

The title compound was prepared using 2-(4-methyl-thiazol-2-yl)-thieno[2,3-d]pyrimidin-4-ylamine in place of and 2-(5-methyl-furan-2-yl)-thieno[2,3-d]pyrimidin-4-ylamine as described in Example 1.

### Example 9

### Step c

### 2-(4-Methyl-thiazol-2-yl)-6-(1-phenyl-vinyl)-thieno[2,3-d]pyrimidin-4-ylamine

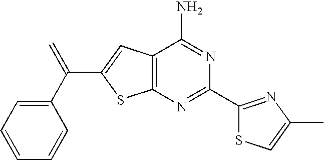

A dioxane (8.0 mL)/water (2.0 mL) solution of 6-bromo-2-(4-methyl-thiazol-2-yl)-thieno[2,3-d]pyrimidin-4-ylamine (252 mg, 0.77 mmol), 1-phenylvinylboronic acid (171 mg, 1.20 mmol), Pd(dppf)Cl₂ (63 mg, 0.08 mmol), and K₂CO₃ (213 mg, 1.54 mmol) was heated to 80° C. After 18 h the mixture was diluted with EtOAc and the solution was washed with water and brine, dried (Na₂SO₄), concentrated and purified via column chromatography to give 131 mg of the title compound. ¹H NMR (Acetone, 400 MHz): δ=7.55-7.60 (m, 2H), 7.50-7.55 (m, 3H), 7.40 (s, 1H), 7.35 (d, J=110 Hz, 1H), 7.15 (br. s, 1H), 5.80 (s, 1H), 5.70 (s, 1H), 2.55 ppm (d, J=1.0 Hz, 3H); MS m/e 351 (M+H).

### Example 10

### 2-(4-Methyl-thiazol-2-yl)-6-(1-phenyl-cyclopropyl)-thieno[2,3-d]pyrimidin-4-ylamine

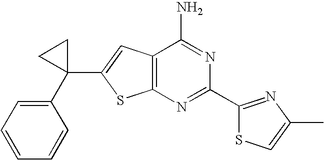

Solid t-BuOK (67 mg, 0.60 mmol) was added to a DMSO solution (0.7 mL) of (CH₃)₃SOI (121 mg, 0.55 mmol). After 30 min a THF solution (2 mL) of 2-(4-methyl-thiazol-2-yl)-6-(1-phenyl-vinyl)-thieno[2,3-d]pyrimidin-4-ylamine (85 mg, 0.24 mmol, prepared as described in Example 9) was added. After 16 h the mixture was diluted with EtOAc and the organic layer was washed with water and brine, dried (Na₂SO₄), and dry packed onto silica gel. Column chromatography gave 19 mg of the title compound. ¹H NMR (CHLOROFORM-d, 300 MHz): δ=7.28-7.44 (m, 5H), 7.00 (s, 1H), 6.61 (s, 1H), 5.31 (br. s, 2H), 2.55 (s, 3H), 1.48 ppm (d, J=5.3 Hz, 4H); MS m/e 365 (M+H).

### Example 11

### [4-Amino-2-(5-methyl-furan-2-yl)-thieno[2,3-d]pyrimidin-6-yl]-(2-methoxy-phenyl)-methanol

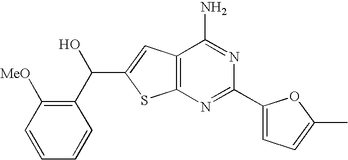

The title compound was prepared using 2-methoxyphenylmagnesium bromide in place of phenylmagnesium bromide as described in Example 3. ¹H NMR (CHLOROFORM-d, 400 MHz): δ=7.30-7.40 (m, 2H), 7.14 (d, J=3.0 Hz, 1H), 6.97-7.04 (m, 1H), 6.91-6.96 (m, 1H), 6.78 (d, J=1.3 Hz, 1H), 6.17 (d, J=6.6 Hz, 1H), 6.13 (dd, J=3.3, 1.0 Hz, 1H), 5.22 (br. s, 2H), 3.84 (s, 3H), 3.60 (d, J=7.1 Hz, 1H), 2.43 ppm (s, 3H); MS m/e 368 (M+H).

### Example 12

### 6-(2-Methoxy-benzyl)-2-(5-methyl-furan-2-yl)-thieno[2,3-d]pyrimidin-4-ylamine

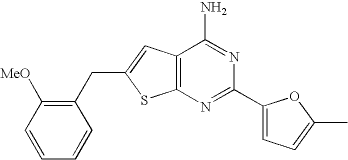

The title compound was prepared using 2-methoxybenzylzinc bromide in place of benzylzinc bromide as described in Example 1. ¹H NMR (CHLOROFORM-d, 300 MHz): δ=7.19-7.30 (m, 2H), 7.13 (d, J=3.4 Hz, 1H), 6.86-6.97 (m, 2H), 6.70 (s, 1H), 6.13 (d, J=2.6 Hz, 1H), 5.14 (br. s, 2H), 4.17 (s, 2H), 3.85 (s, 3H), 2.44 ppm (s, 3H); MS m/e 352 (M+H).

### Example 13

### 6-Benzyl-2-pyridin-3-yl-thieno[2,3-d]pyrimidin-4-ylamine

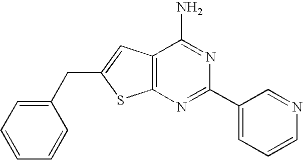

The title compound was prepared using pyridine-3-carbonitrile in place of 5-methyl-furan-2-carbonitrile as described in Example 1. ¹H NMR (Acetone, 300 MHz): δ=9.43 (s, 1H), 8.32-8.63 (m, 2H), 7.31 (dd, J=7.9, 4.9 Hz, 1H), 7.23 (d, J=4.5 Hz, 4H), 7.10-7.20 (m, 2H), 6.80 (br. s, 2H), 4.14 ppm (s, 2H); MS m/e 319 (M+H).

### Example 14

### 6-Benzyl-2-(4-methyl-thiazol-2-yl)-thieno[2,3-d]pyrimidin-4-ylamine

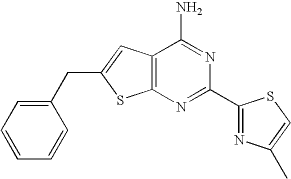

The title compound was prepared using 6-bromo-2-(4-methyl-thiazol-2-yl)-thieno[2,3-d]pyrimidin-4-ylamine (an intermediate prepared in Example 9), in place of 6-bromo-2-(5-methyl-furan-2-yl)-thieno[2,3-d]pyrimidin-4-ylamine as described in Example 1. ¹H NMR (DMSO-d₆, 300 MHz): δ=7.67 (s, 1H), 7.40 (s, 1H), 7.24-7.37 (m, 5H), 4.23 (s, 2H), 3.30 ppm (s, 3H); MS m/e 339 (M+H).

### Example 15

### 6-(2-Methoxy-benzyl)-2-(4-methyl-thiazol-2-yl)-thieno[2,3-d]pyrimidin-4-ylamine

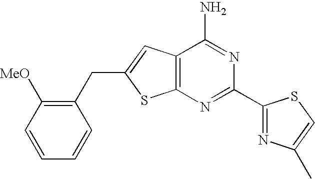

The title compound was prepared using 2-methoxybenzylzinc bromide and 6-bromo-2-(4-methyl-thiazol-2-yl)-thieno[2,3-d]pyrimidin-4-ylamine (an intermediate prepared in Example 9), in place of benzylzinc bromide and 6-bromo-2-(5-methyl-furan-2-yl)-thieno[2,3-d]pyrimidin-4-ylamine, respectively, as described in Example 1. ¹H NMR (CHLOROFORM-d, 400 MHz): δ=7.22 (dd, J=7.3, 1.5 Hz, 2H), 6.97-7.04 (m, 1H), 6.87-6.97 (m, 2H), 6.76 (s, 1H), 5.29 (br. s, 2H), 4.19 (s, 2H), 3.86 (s, 3H), 2.57 ppm (s, 3H); MS m/e 369 (M+H).

### Example 16

### (±)-2-(5-Methyl-furan-2-yl)-6-(1-phenyl-ethyl)-thieno[2,3-d]pyrimidin-4-ylamine

### Example 16

### Step a

### (±)-2-Amino-5-(1-phenyl-ethyl)-thiophene-3-carbonitrile

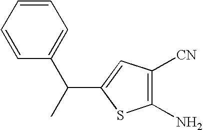

Triethylamine (7.07 mL, 50.8 mmol, 0.6 equiv) was added dropwise by addition funnel to an ice-cold mixture of sulfur (2.71 g, 84.5 mmol, 1 equiv) and racemic 3-phenylbutyraldehyde (15.1 mL, 101.5 mmol, 1.2 equiv) in DMF (17 mL). The resulting suspension was stirred at room temperature for 50 min. After cooling to 0° C., a solution of malononitrile (5.59 g, 84.5 mmol, 1 equiv) in DMF (11 mL) was added. The resulting suspension was stirred at room temperature for 40 min, then was poured into 200 mL stirred ice water, resulting in a tarry suspension. Methanol (100 mL) was added and the suspension was heated to boiling, hot-filtered, and allowed to cool. The resulting brown precipitate was collected by vacuum filtration and was washed with water. Column chromatography gave 579 mg of the title compound.

### Example 16

### Step b

### (±)-2-(5-Methyl-furan-2-yl)-6-(1-phenyl-ethyl)-thieno[2,3-d]pyrimidin-4-ylamine

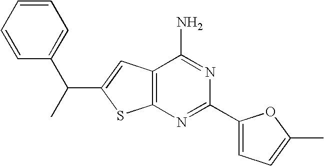

The title compound was prepared using (±)-2-amino-5-(1-phenyl-ethyl)-thiophene-3-carbonitrile in place of 2-amino-thiophene-3-carbonitrile as described in Example 1. 1H NMR (300 MHz, CHLOROFORM-D) δ ppm 7.24-7.37 (m, 5H), 7.14 (d, J=3.4 Hz, 1H), 6.70 (d, J=1.5 Hz, 1H), 6.14 (dd, J=3.4, 0.8 Hz, 1H), 5.22 (s, 2H), 4.35 (q, J=7.0 Hz, 1H), 2.44 (s, 3H), 1.75 (d, J=7.2 Hz, 3H); MS m/e 336 (M+H).

### Example 17

### (±)-2-(4-Methyl-thiazol-2-yl)-6-(1-phenyl-ethyl)-thieno[2,3-d]pyrimidin-4-ylamine

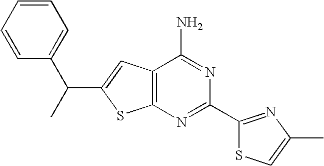

The title compound was prepared using (±)-2-amino-5-(1-phenyl-ethyl)-thiophene-3-carbonitrile (an intermediate prepared in Example 16) and 4-methylthiazole-2-carbonitrile in place of 2-amino-thiophene-3-carbonitrile and 5-methyl-furan-2-carbonitrile, respectively, as described in Example 1. 1H NMR (300 MHz, DMSO-D6) δ ppm 7.67 (br s, 2H), 7.45 (d, J=0.8 Hz, 1H), 7.23-7.39 (m, 6H), 4.44 (q, J=6.9 Hz, 1H), 2.42 (s, 3H), 1.68 (d, J=7.2 Hz, 3H); MS m/e 353 (M+H).

### Example 18

### 5-(4-Amino-6-benzyl-thieno[2,3-d]pyrimidin-2-yl)-furan-2-carbonitrile

### Example 18

### Step a

### 2-Amino-5-benzyl-thiophene-3-carbonitrile

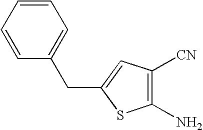

The title compound was prepared using 3-phenyl-propionaldehyde in place of 3-phenylbutyraldehyde as described in Example 16.

### Example 18

### Step b

### 5-(4-Amino-6-benzyl-thieno[2,3-d]pyrimidin-2-yl)-furan-2-carbonitrile

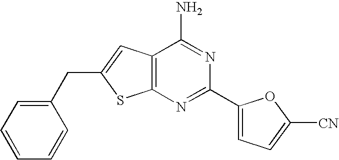

The title compound was prepared using 2-amino-5-benzyl-thiophene-3-carbonitrile and 2,5-dicyanofuran in place of 2-amino-thiophene-3-carbonitrile and 5-methyl-furan-2-carbonitrile, respectively, as described in Example 1. 1H NMR (400 MHz, CHLOROFORM-D) δ ppm 7.34-7.39 (m, 2H), 7.25-7.32 (m, 4H), 7.20 (d, J=3.7 Hz, 1H), 6.76 (s, 1H), 5.34 (br s, 2H), 4.22 (s, 2H); MS m/e 333 (M+H).

### Example 19

### 6-Benzyl-2-oxazol-5-yl-thieno[2,3-d]pyrimidin-4-ylamine

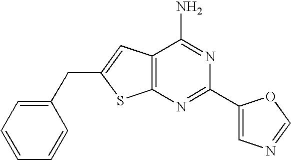

The title compound was prepared using 2-amino-5-benzyl-thiophene-3-carbonitrile (an intermediate prepared in Example 18) and 5-oxazolecarbonitrile in place of 2-amino-thiophene-3-carbonitrile and 5-methyl-furan-2-carbonitrile, respectively, as described in Example 1. 1H NMR (400 MHz, CHLOROFORM-D) δ ppm 8.00 (s, 1H), 7.83 (s, 1H), 7.27-7.38 (m, 5H), 6.74 (s, 1H), 5.24 (br s, 2H), 4.21 (s, 2H); MS m/e 309 (M+H).

### Example 20

### 6-Benzyl-2-(1-methyl-1H-imidazol-4-yl)-thieno[2,3-d]pyrimidin-4-ylamine

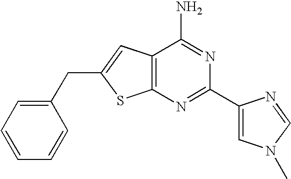

The title compound was prepared using 2-amino-5-benzyl-thiophene-3-carbonitrile (an intermediate prepared in Example 18) and 1-methyl-1H-imidazole-4-carbonitrile in place of 2-amino-thiophene-3-carbonitrile and 5-methyl-furan-2-carbonitrile, respectively, as described in Example 1. 1H NMR (300 MHz, DMSO-D6) δ ppm 7.68 (d, J=1.5 Hz, 1H), 7.60 (d, J=1.1 Hz, 1H), 7.22-7.36 (m, 8H), 4.18 (s, 2H), 3.69 (s, 3H); MS m/e 322 (M+H)

### Example 21

### (±)-2-Oxazol-2-yl-6-(1-phenyl-ethyl)-thieno[2,3-d]pyrimidin-4-ylamine

### Example 21

### Step a

### Oxazole-2-carboxylic acid amide

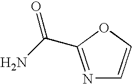

Oxazole-2-carboxylic acid ethyl ester (1.6 g, 11.4 mmol) was suspended in concentrated NH₄OH (32 mL) and stirred vigorously. After 26 h the precipitate was collected by vacuum filtration, affording 1.1 g of the title compound that was used without further purification.

### Example 21

### Step b

### Oxazole-2-carbonitrile

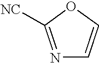

Neat POCl₃ (1.12 mL, 12.3 mmol) was added to a pyridine solution (17 mL) of oxazole-2-carboxylic acid amide (982 mg, 8.8 mmol). After 4 h the mixture was cooled to 0° C. and taken to pH 3 with concentrated aqueous HCl. The aqueous mixture was extracted with Et₂O and the combined extracts were washed with water then brine, dried (Mg₂SO₄), concentrated and used without further purification to give 478 mg of 5-cyclopropyl-furan-2-carbonitrile.

The residue contained water, and was therefore dissolved in CH₂Cl₂, dried (Na₂SO₄), and concentrated to give 573 mg (70% pure, 30% pyridine) that was used without further purification.

### Example 21

### Step c

### Example 21

### (±)-2-Oxazol-2-yl-6-(1-phenyl-ethyl)-thieno[2,3-d]pyrimidin-4-ylamine

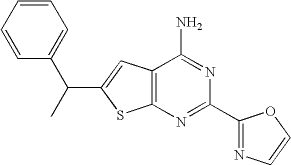

The title compound was prepared using (±)-2-amino-5-(1-phenyl-ethyl)-thiophene-3-carbonitrile (an intermediate prepared in Example 16) and 2-oxazolecarbonitrile in place of 2-amino-thiophene-3-carbonitrile and 5-methyl-furan-2-carbonitrile, respectively, as described in Example 1. 1H NMR (300 MHz, CD₃OD) δ ppm 8.08 (d, J=0.8 Hz, 1H), 7.39 (s, 1H), 7.30-7.34 (m, 5H), 7.21-7.28 (m, 1H), 4.43 (q, J=6.9 Hz, 1H), 1.76 (d, J=7.2 Hz, 3H); MS m/e 323 (M+H).

### Example 22

### 2-(5-Methyl-furan-2-yl)-6-phenethyl-thieno[2,3-d]pyrimidin-4-ylamine

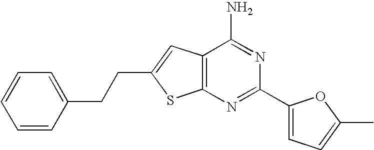

The title compound was prepared using phenethylzinc bromide in place of benzylzinc bromide as described in Example 1. ¹H NMR (CHLOROFORM-d, 300 MHz): δ=7.27-7.33 (m, 2H), 7.18-7.25 (m, 3H), 7.15 (d, J=3.0 Hz, 1H), 6.73 (s, 1H), 6.15 (d, J=2.6 Hz, 1H), 5.17 (br. s, 2H), 3.12-3.26 (m, 2H), 2.93-3.10 (m, 2H), 2.45 ppm (s, 3H); MS m/e 336 (M+H).

### Example 23

### 6-Benzyl-2-(5-cyclopropyl-furan-2-yl)-thieno[2,3-d]pyrimidin-4-ylamine

### Example 23

### Step a

### 5-Cyclopropyl-furan-2-carboxylic acid methyl ester

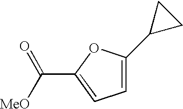

Solid cyclopropylboronic acid (575 mg, 6.7 mmol) was added to a toluene (22 mL)/water (1.1 mL) solution of 5-bromo-furan-2-carboxylic acid methyl ester (980 mg, 4.8 mmol), Pd(OAc)₂ (54 mg, 0.2 mmol), P(Cy)₃ (135 mg, 0.5 mmol), and K₃PO₄ (3.6 g, 16.8 mmol). The resulting mixture was heated to 90° C. After 5 h the mixture was cooled, filtered and extracted with EtOAc. The combined organic extracts were washed with water and brine, dried (Na₂SO₄), concentrated and purified via column chromatography to give 650 mg of 5-cyclopropyl-furan-2-carboxylic acid methyl ester.

### Example 23

### Step b

### 5-Cyclopropyl-furan-2-carboxylic acid amide

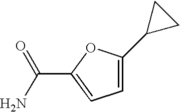

5-cyclopropyl-furan-2-carboxylic acid methyl ester (650 mg, 3.9 mmol) was suspended in concentrated NH₄OH (20 mL) and stirred vigorously. After 16 h the mixture was diluted with water and the aqueous phase was extracted with EtOAc. The combined organic extracts were washed with water and brine, dried (Na₂SO₄), concentrated and used without further purification to give 550 mg of 5-cyclopropyl-furan-2-carboxylic acid amide.

### Example 23

### Step c

### 5-Cyclopropyl-furan-2-carbonitrile

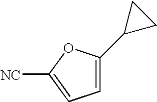

Neat POCl₃ (0.48 mL, 5.1 mmol) was added to a pyridine solution (9 mL) of 5-cyclopropyl-furan-2-carboxylic acid amide (550 mg, 3.6 mmol). After 2 h the mixture was cooled to 0° C. and taken to pH 4.5 with concentrated aqueous HCl. The aqueous mixture was extracted with Et₂O and the combined extracts were washed with brine, dried (Na₂SO₄), concentrated and used without further purification to give 478 mg of 5-cyclopropyl-furan-2-carbonitrile.

### Example 23

### Step d

### 6-Benzyl-2-(5-cyclopropyl-furan-2-yl)-thieno[2,3-d]pyrimidin-4-ylamine

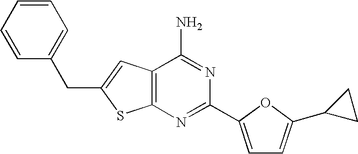

The title compound was prepared using 2-amino-5-benzyl-thiophene-3-carbonitrile (an intermediate prepared in Example 18) and 5-cyclopropyl-furan-2-carbonitrile in place of 2-amino-thiophene-3-carbonitrile and 5-methyl-furan-2-carbonitrile, respectively, as described in Example 1. ¹H NMR (CHLOROFORM-d, 300 MHz): δ=7.27-7.37 (m, 5H), 7.13 (d, J=3.4 Hz, 1H), 6.68 (s, 1H), 6.02 (d, J=2.6 Hz, 1H), 5.23 (br. s, 2H), 4.18 (s, 2H), 2.05 (t, J=5.1 Hz, 1H), 0.90-0.96 (m, 2H), 0.79-0.84 ppm (m, 2H); MS m/e 348 (M+H)

### Example 24

### 6-(4-Amino-6-benzyl-thieno[2,3-d]pyrimidin-2-yl)-pyridine-2-carbonitrile

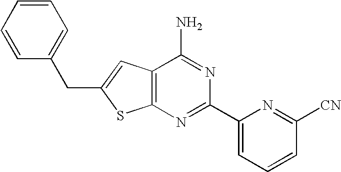

The title compound was prepared using 2-amino-5-benzyl-thiophene-3-carbonitrile (an intermediate prepared in Example 18) and pyridine-2,6-dicarbonitrile in place of 2-amino-thiophene-3-carbonitrile and 5-methyl-furan-2-carbonitrile, respectively, as described in Example 1. ¹H NMR (DMSO-d₆, 300 MHz): δ=8.55-8.64 (m, 1H), 8.07-8.22 (m, 2H), 7.76 (br. s, 2H), 7.30-7.41 (m, 5H), 6.77 (s, 1H), 4.26 ppm (s, 2H); MS m/e 344 (M+H)

### Example 25

### 6-Benzyl-2-pyrimidin-2-yl-thieno[2,3-d]pyrimidin-4-ylamine hydrochloride

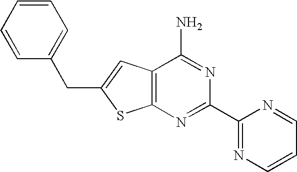

The title compound was prepared using 2-amino-5-benzyl-thiophene-3-carbonitrile (an intermediate prepared in Example 18) and pyrimidine-2-carbonitrile in place of 2-amino-thiophene-3-carbonitrile and 5-methyl-furan-2-carbonitrile, respectively, as described in Example 1. ¹H NMR (DMSO-d₆, 300 MHz): δ=9.01 (d, J=4.9 Hz, 2H), 7.66 (t, J=4.7 Hz, 1H), 7.47 (s, 1H), 7.23-7.42 (m, 5H), 4.29 ppm (s, 2H); MS m/e 320 (M+H)

### Example 26

### 6-Benzyl-2-(5-tert-butyl-thiophen-2-yl)-thieno[2,3-d]pyrimidin-4-ylamine

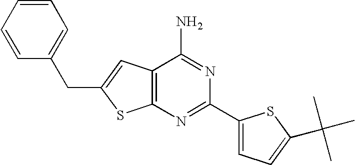

The title compound was prepared using 2-amino-5-benzyl-thiophene-3-carbonitrile (an intermediate prepared in Example 18) and 5-tert-butyl-thiophene-2-carbonitrile in place of 2-amino-thiophene-3-carbonitrile and 5-methyl-furan-2-carbonitrile, respectively, as described in Example 1. ¹H NMR (CHLOROFORM-d, 300 MHz): δ=7.28-7.36 (m, 5H), 6.85 (d, J=3.8 Hz, 1H), 6.70 (s, 1H), 6.41 (s, 1H), 4.17 (s, 2H), 3.91 (s, 2H), 1.41 ppm (s, 9H); MS m/e 380 (M+H)

### Example 27

### 6-Benzyl-2-(5-isopropyl-furan-2-yl)-thieno[2,3-d]pyrimidin-4-ylamine

### Example 27

### Step a

### 5-Isopropyl-furan-2-carboxylic acid methyl ester

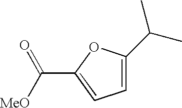

A 0.5 M THF solution (7.3 mL, 3.6 mmol) of isopropylzinc bromide was added to a THF solution (2 mL) of 5-bromo-furan-2-carboxylic acid methyl ester (250 mg, 1.2 mmol) and Pd(dppf)Cl₂ (98 mg, 0.1 mmol) and the resulting mixture was heated to 70° C. After 15 h the mixture was cooled, water was added and the aqueous phase was extracted with EtOAc. The combined organic extracts were washed with water and brine, dried (Na₂SO₄), concentrated and purified via column chromatography to give 150 mg of 5-isoopropyl-furan-2-carboxylic acid methyl ester.

### Example 27

### Step b

### 5-Isopropyl-furan-2-carboxylic acid amide

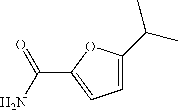

The title compounds was prepared using 5-isopropyl-furan-2-carboxylic acid methyl ester in place of 5-cyclopropyl-furan-2-carboxylic acid methyl ester as described in example 23.

### Example 27

### Step c

### 5-Isopropyl-furan-2-carbonitrile

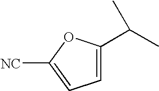

The title compound was prepared using 5-isopropyl-furan-2-carboxylic acid amide in place of 5-cyclopropyl-furan-2-carbonitrile. as described in example 23.

### Example 27

### Step d

### 6-Benzyl-2-(5-isopropyl-furan-2-yl)-thieno[2,3-d]pyrimidin-4-ylamine

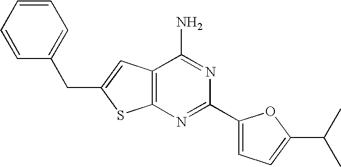

The title compound was prepared using 2-amino-5-benzyl-thiophene-3-carbonitrile (an intermediate prepared in Example 18) and 5-isopropyl-furan-2-carbonitrile in place of 2-amino-thiophene-3-carbonitrile and 5-methyl-furan-2-carbonitrile, respectively, as described in Example 1. ¹H NMR (CHLOROFORM-d, 400 MHz): δ=7.26-7.38 (m, 5H), 7.15 (d, J=3.4 Hz, 1H), 6.68 (s, 1H), 6.13 (dd, J=3.4, 1.0 Hz, 1H), 5.21 (br. s, 2H), 4.20 (s, 2H), 3.04-3.18 (m, 1H), 1.24-1.36 ppm (m, 6H); MS m/e 350 (M+H)

### Example 28

### 6-Benzyl-2-(2-methyl-thiazol-4-yl)-thieno[2,3-d]pyrimidin-4-ylamine

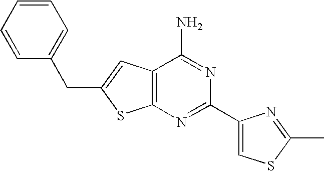

The title compound was prepared using 2-amino-5-benzyl-thiophene-3-carbonitrile (an intermediate prepared in Example 18) and 2-methyl-thiazole-4-carbonitrile in place of 2-amino-thiophene-3-carbonitrile and 5-methyl-furan-2-carbonitrile, respectively, as described in Example 1. ¹H NMR (CHLOROFORM-d, 300 MHz): δ=8.08 (s, 1H), 7.27-7.39 (m, 5H), 6.72 (s, 1H), 5.25 (br. s, 2H), 4.20 (s, 2H), 2.82 ppm (s, 3H); MS m/e 339 (M+H).

### Example 29

### 6-[2-(4-Fluoro-phenyl)-ethyl]-2-(5-methyl-furan-2-yl)-thieno[2,3-d]pyrimidin-4-ylamine

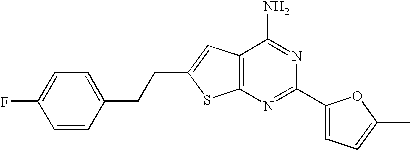

The title compound was prepared using 4-fluorophenethylzinc bromide in place of benzylzinc bromide as described in Example 1. ¹H NMR (CHLOROFORM-d, 300 MHz): δ=7.08-7.19 (m, 3H), 6.90-7.02 (m, 2H), 6.70 (s, 1H), 6.15 (d, J=2.3 Hz, 1H), 5.17 (br. s, 2H), 3.11-3.21 (m, 2H), 2.93-3.06 (m, 2H), 2.45 ppm (s, 3H); MS m/e 354 (M+H).

### Example 30

### 6-[2-(4-Fluoro-phenyl)-vinyl]-2-(5-methyl-furan-2-yl)-thieno[2,3-d]pyrimidin-4-ylamine

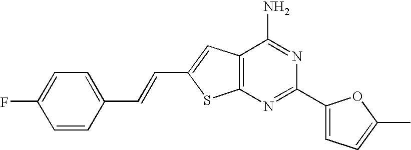

The title compound was prepared using 2-(4-fluorophenyl)vinylboronic acid in place of vinylboronic acid dibutyl ester as described in Example 2. ¹H NMR (CHLOROFORM-d, 300 MHz): δ=7.47 (dd, J=8.7, 5.3 Hz, 2H), 7.19 (d, J=3.0 Hz, 1H), 6.88-7.18 (m, 6H), 6.17 (d, J=2.3 Hz, 1H), 5.25 (br. s, 2H), 2.46 ppm (s, 3H); MS m/e 352 (M+H).

### Example 31

### 5-(4-Amino-6-benzyl-thieno[2,3-d]pyrimidin-2-yl)-nicotinonitrile

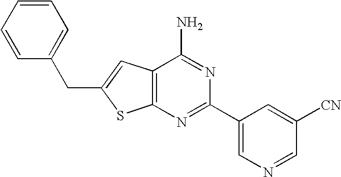

The title compound was prepared using 2-amino-5-benzyl-thiophene-3-carbonitrile (an intermediate prepared in Example 18) and pyridine-3,5-dicarbonitrile in place of 2-amino-thiophene-3-carbonitrile and 5-methyl-furan-2-carbonitrile, respectively, as described in Example 1. ¹H NMR (DMSO-d₆, 300 MHz): δ=9.64 (d, J=1.9 Hz, 1H), 9.09 (d, J=2.3 Hz, 1H), 8.87-8.95 (m, 1H), 7.70 (br. s, 2H), 7.21-7.43 (m, 6H), 4.25 ppm (s, 2H); MS m/e 344 (M+H).

### Example 32

### 6-Benzyl-2-pyrazin-2-yl-thieno[2,3-d]pyrimidin-4-ylamine

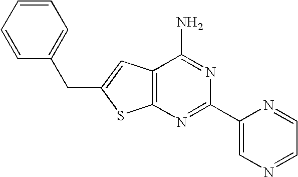

The title compound was prepared using 2-amino-5-benzyl-thiophene-3-carbonitrile (an intermediate prepared in Example 18) and pyrazine-2-carbonitrile in place of 2-amino-thiophene-3-carbonitrile and 5-methyl-furan-2-carbonitrile, respectively, as described in Example 1. ¹H NMR (DMSO-d₆, 300 MHz): δ=9.48 (s, 1H), 8.71-8.84 (m, 2H), 8.04 (br. s, 2H), 7.43 (s, 1H), 7.24-7.41 (m, 5H), 4.28 ppm (s, 2H); MS m/e 320 (M+H).

### Example 33

### 2-(5-Methyl-furan-2-yl)-6-styryl-thieno[2,3-d]pyrimidin-4-ylamine

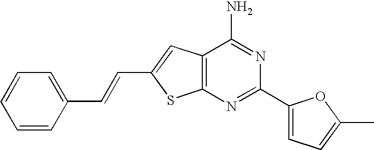

The title compound was prepared using 2-phenylvinylboronic acid in place of vinylboronic acid dibutyl ester as described in Example 2. ¹H NMR (DMSO-d₆, 300 MHz): δ=7.59-7.72 (m, 4H), 7.45-7.57 (m, 2H), 7.39 (t, J=7.7 Hz, 2H), 7.30 (d, J=7.2 Hz, 1H), 7.06 (d, J=3.4 Hz, 1H), 6.95 (d, J=16.2 Hz, 1H), 6.28 (d, J=3.4 Hz, 1H), 2.38 ppm (s, 3H); MS m/e 334 (M+H).

### Example 34

### 6-(2-Chloro-benzyl)-2-pyrimidin-2-yl-thieno[2,3-d]pyrimidin-4-ylamine

### Example 34

### Step a

### 3-(2-Chloro-phenyl)-propionaldehyde

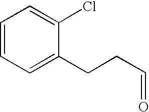

Solid tetrabutylammonium chloride (1.2 g, 4.2 mmol) was added to a DMF solution (5.5 mL) of Pd(OAc)₂ (57 mg, 0.1 mmol), NaHCO₃ (880 mg, 10.5 mmol), 1-chloro-2-iodo-benzene (1.0 g, 4.2 mmol), and allyl alcohol (370 mg, 6.29 mmol) in a sealed tube and the mixture was heated to 45° C. After 22 h at 45° C., the solution was cooled to room temperature; water was added, and the aqueous phase was extracted with ether, dried (Na₂SO₄) and concentrated to give 0.66 g of the title compound that was used in the next step without further purification.

### Example 34

### Step b

### 2-Amino-5-(2-chloro-benzyl)-thiophene-3-carbonitrile

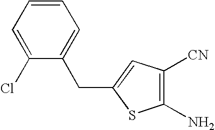

The title compound was prepared using 3-(2-Chloro-phenyl)-propionaldehyde in place of 3-phenylbutyraldehyde as described in Example 16.

### Example 34

### Step c

### 6-(2-Chloro-benzyl)-2-pyrimidin-2-yl-thieno[2,3-d]pyrimidin-4-ylamine

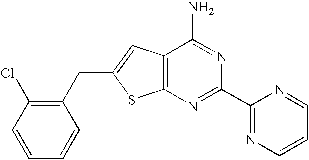

The title compound was prepared using 2-amino-5-(2-chloro-benzyl)-thiophene-3-carbonitrile and pyrimidine-2-carbonitrile in place of 2-amino-thiophene-3-carbonitrile and 5-methyl-furan-2-carbonitrile, respectively, as described in Example 1. ¹H NMR (300 MHz, CD₃OD) δ 9.02 (d, J=4.90 Hz, 2H), 7.64 (t, J=4.90 Hz, 1H), 7.41-7.54 (m, 2H), 7.24-7.41 (m, 3H), 4.45 (s, 2H); MS m/e 354 (M+H).

### Example 35

### 6-[4-Amino-6-(2-chloro-benzyl)-thieno[2,3-d]pyrimidin-2-yl]-pyridine-2-carbonitrile

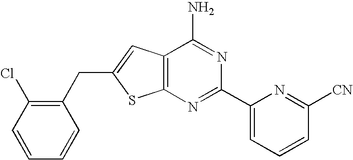

The title compound was prepared using 2-amino-5-(2-chloro-benzyl)-thiophene-3-carbonitrile (an intermediate prepared in Example 34) and pyridine-2,6-dicarbonitrile in place of 2-amino-thiophene-3-carbonitrile and 5-methyl-furan-2-carbonitrile, respectively, as described in Example 1. ¹H NMR (300 MHz, CD₃OD) δ 8.72 (d, J=7.16 Hz, 1H), 8.10 (t, J=7.91 Hz, 1H), 7.91 (d, J=7.54 Hz, 1H), 7.37-7.52 (m, 2H), 7.24-7.37 (m, 2H), 7.20 (s, 1H), 4.39 (s, 2H); MS m/e 378 (M+H).

### Example 36

### 6-(2-Chloro-benzyl)-2-(4-methyl-thiazol-2-yl)-thieno[2,3-d]pyrimidin-4-ylamine

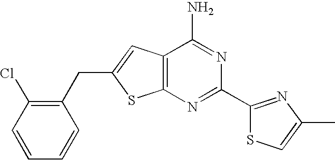

The title compound was prepared using 2-amino-5-(2-chloro-benzyl)-thiophene-3-carbonitrile (an intermediate prepared in Example 34) and 2-methyl-thiazole-4-carbonitrile in place of 2-amino-thiophene-3-carbonitrile and 5-methyl-furan-2-carbonitrile, respectively, as described in Example 1. ¹H NMR (300 MHz, CHLOROFORM-d, CD₃OD) δ 7.33-7.48 (m, 2H), 7.19-7.34 (m, 2H), 7.15 (d, J=6.03 Hz, 2H), 4.36 (s, 2H), 2.53 (s, 3H); MS m/e 373 (M+H).

### Example 37

### 6-(2-Chloro-benzyl)-2-pyrazin-2-yl-thieno[2,3-d]pyrimidin-4-ylamine

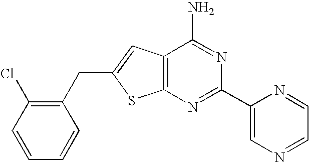

The title compound was prepared using 2-amino-5-(2-chloro-benzyl)-thiophene-3-carbonitrile (an intermediate prepared in Example 34) and pyrazine-2-carbonitrile in place of 2-amino-thiophene-3-carbonitrile and 5-methyl-furan-2-carbonitrile, respectively, as described in Example 1. ¹H NMR (300 MHz, DMSO-d₆) δ 9.45 (d, J=1.51 Hz, 1H), 8.61-8.80 (m, 2H), 7.68 (s, 2H), 7.51 (dd, J=2.45, 6.22 Hz, 2H), 7.24-7.43 (m, 3H), 4.35 (s, 2H); MS m/e 354 (M+H).

### Example 38

### 6-(2-Chloro-benzyl)-2-(5-methyl-furan-2-yl)-thieno[2,3-d]pyrimidin-4-ylamine

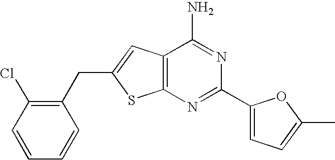

The title compound was prepared using 2-amino-5-(2-chloro-benzyl)-thiophene-3-carbonitrile (an intermediate prepared in Example 34) in place of 2-amino-thiophene-3-carbonitrile as described in Example 1. ¹H NMR (300 MHz, DMSO-d₆) δ 7.41-7.58 (m, 4H), 7.27-7.42 (m, 2H), 7.21 (s, 1H), 6.99 (d, J=3.39 Hz, 1H), 6.24 (d, J=2.26 Hz, 1H), 4.29 (s, 2H), 2.35 (s, 3H); MS m/e 356 (M+H).

### Example 39

### 2-(5-Difluoromethyl-furan-2-yl)-6-(2-methoxy-benzyl)-thieno[2,3-d]pyrimidin-4-ylamine

### Example 39

### Step a

### 5-Difluoromethyl-furan-2-carbonitrile

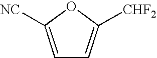

To a solution of Et₂NSF₃ (2.8 mL, 21.4 mmol) and CH₂Cl₂ (10 mL) at 4° C. was added a solution of 5-formyl-furan-2-carbonitrile (2.44 g, 20.2 mmol; W. Hoyle and G. P. Roberts, J. Med. Chem. 1973, 16, 709) in CH₂Cl₂ (10 mL). After 30 min at 4° C., saturated aqueous NaHCO₃ was added, the layers were separated and the aqueous layer was extracted with CH₂Cl₂. The combined organics were dried (Na₂SO₄) and concentrated to give 2.15 g of 5-difluoromethyl-furan-2-carbonitrile that was used without further purification.

### Example 39

### Step b

### 2-(5-Difluoromethyl-furan-2-yl)-6-(2-methoxy-benzyl)-thieno[2,3-d]pyrimidin-4-ylamine

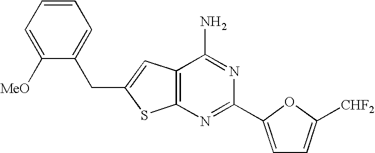

The title compound was prepared using 2-methoxybenzylzinc bromide and 5-difluoromethyl-furan-2-carbonitrile in place of benzylzinc bromide and 5-methyl-furan-2-carbonitrile, respectively, as described in Example 1. ¹H NMR (400 MHz, CDCl₃) δ=7.20-7.31 (m, 3H), 6.88-6.95 (m, 2H), 6.79-6.82 (m, 1H), 6.78 (s, 1H), 6.76 (t, J=54.4 Hz, 1H), 5.86 (br. s, 2H), 4.20 (s, 2H), 3.86 (s, 3H); MS m/e 388 (M+H).

### Example 40

### [4-Amino-2-(2-methoxy-pyridin-4-yl)-thieno[2,3-d]pyrimidin-6-yl]-phenyl-methanol

### Example 40

### Step a

### 2-(2-Methoxy-pyridin-4-yl)-thieno[2,3-d]pyrimidin-4-ylamine

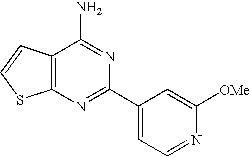

The title compound was prepared using 2-methoxy-isonicotinonitrile and in place of 5-methyl-furan-2-carbonitrile as described in Example 1.

### Example 40

### Step b

### [2-(2-Methoxy-pyridin-4-yl)-thieno[2,3-d]pyrimidin-4-yl]-bis-carbamic acid tert-butyl ester

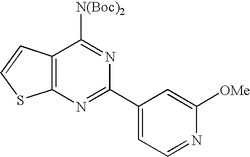

Solid DMAP (100 mg, 0.82 mmol) was added to a THF solution (20 mL) of 2-(2-methoxy-pyridin-4-yl)-thieno[2,3-d]pyrimidin-4-ylamine (2.0 g, 8.0 mmol) and (Boc)₂O (4.4 g, 20.2 mmol). After 2 h the mixture was concentrated in vacuo, and the resulting solid was diluted with CH₂Cl₂, filtered, and the filtrate was concentrated and purified by column chromatography to give 3.0 g of the title compound.

### Example 40

### Step c

### [4-Amino-2-(2-methoxy-pyridin-4-yl)-thien o[2,3-d]pyrimidin-6-yl]-phenyl-methanol

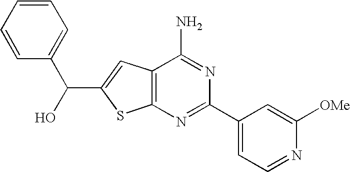

A 1.8 M LDA solution (0.30 mL, 0.54 mmol) was added to a −78° C. THF solution (2.5 mL) of [2-(2-methoxy-pyridin-4-yl)-thieno[2,3-d]pyrimidin-4-yl]-bis-carbamic acid tert-butyl ester (223 mg, 0.49 mmol). After 8 min, neat benzaldehyde (77 mg, 0.73 mmol) was added and the mixture was allowed to warm to −20° C. over 40 min. Saturated aqueous NH₄Cl was added and the layers were separated. The aqueous layer was extracted with CH₂Cl₂ and the combined organics were dried (Na₂SO₄), concentrated, and purified by column chromatography to give 110 mg of [6-(Hydroxy-phenyl-methyl)-2-(2-methoxy-pyridin-4-yl)-thieno[2,3-d]pyrimidin-4-yl]-bis-carbamic acid tert-butyl ester. Neat TFA (0.3 mL) was added to a CH₂Cl₂ solution (0.8 mL) of [6-(Hydroxy-phenyl-methyl)-2-(2-methoxy-pyridin-4-yl)-thieno[2,3-d]pyrimidin-4-yl]-bis-carbamic acid tert-butyl ester (27 mg). After 1 h the mixture was concentrated and the resulting solid was partitioned between CH₂Cl₂ and saturated aqueous NaHCO₃. The organic phase was separated, dried (Na₂SO₄), and concentrated to provide 8 mg of [4-amino-2-(2-methoxy-pyridin-4-yl)-thieno[2,3-d]pyrimidin-6-yl]-phenyl-methanol. ¹H NMR (300 MHz, Acetone-d₆) δ=8.22 (d, J=5.3 Hz, 1H), 7.87 (d, J=5.7 Hz, 1H), 7.69 (s, 1H), 7.53 (d, J=7.5 Hz, 2H), 7.32-7.44 (m, 3H), 7.30 (s, 1H), 6.95 (br. s, 2H), 6.12 (d, J=4.1 Hz, 1H), 5.55 (d, J=4.5 Hz, 1H), 3.92 (s, 3H); MS m/e 365 (M+H).

### Example 41

### [4-Amino-2-(5-difluoromethyl-furan-2-yl)-thieno[2,3-d]pyrimidin-6-yl]-phenyl-methanol

### Example 41

### Step a

### 2-(5-Difluoromethyl-furan-2-yl)-6-methyl-thieno[2,3-d]pyrimidin-4-ylamine

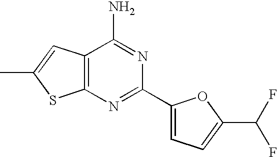

The title compound was prepared using 2-amino-5-methyl-thiophene-3-carbonitrile and 5-difluoromethyl-furan-2-carbonitrile (an intermediate prepared in Example 40) in place of 2-amino-thiophene-3-carbonitrile and 5-methyl-furan-2-carbonitrile, respectively, as described in Example 1.

### Example 41

### Step b

### 4-Amino-2-(5-difluoromethyl-furan-2-yl)-thieno[2,3-d]pyrimidine-6-carbaldehyde

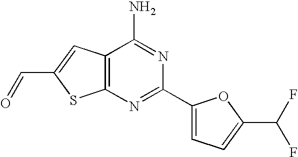

Solid SeO₂ (2.10 g, 18.9 mmol) was added to a dioxane slurry (20 mL) of 2-(5-difluoromethyl-furan-2-yl)-6-methyl-thieno[2,3-d]pyrimidin-4-ylamine (1.77 g, 6.30 mmol) and Celite (0.75 g) and the mixture was heated to 110° C. After 21 h the slurry was dry packed onto silica gel. Column chromatography gave 900 mg of 4-amino-2-(5-difluoromethyl-furan-2-yl)-thieno[2,3-d]pyrimidine-6-carbaldehyde.

### Example 41

### Step c

### [4-Amino-2-(5-difluoromethyl-furan-2-yl)-thieno[2,3-d]pyrimidin-6-yl]-phenyl-methanol

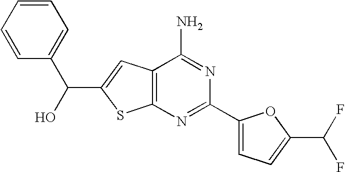

The title compound was prepared using 4-amino-2-(5-difluoromethyl-furan-2-yl)-thieno[2,3-d]pyrimidine-6-carbaldehyde in place of 4-amino-2-(5-methyl-furan-2-yl)-thieno[2,3-d]pyrimidine-6-carbaldehyde as described in Example 2. ¹H NMR (300 MHz, Acetone-d₆) δ=7.44-7.60 (m, 2H), 7.11-7.44 (m, 6H), 6.99 (t, J=53.7 Hz, 1H), 6.87-7.07 (m, 2H), 6.10 (br. s, 1H), 5.54 (br. s, 1H); MS m/e: 374 (M+H).

### Example 42

### 4-{4-Amino-6-[hydroxy-(2-methoxy-phenyl)-methyl]-thieno[2,3-d]pyrimidin-2-yl}-pyridine-2-carbonitrile

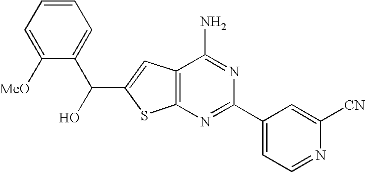

The title compound was prepared using pyridine-2,4-dicarbonitrile and 2-methoxybezaldehyde in place of 2-methoxy-isonicotinonitrile and benzaldehyde, respectively, as described in Example 40. ¹H NMR (300 MHz, Acetone-d₆) δ=8.83 (d, J=4.9 Hz, 1H), 8.69 (s, 1H), 8.54 (dd, J=1.7, 5.1 Hz, 1H), 7.62 (dd, J=1.5, 7.5 Hz, 1H), 7.24-7.39 (m, 2H), 6.95-7.18 (m, 4H), 6.42 (d, J=4.9 Hz, 1H), 5.35 (d, J=4.9 Hz, 1H), 3.88 (s, 3H); MS m/e: 390 (M+H).

### Example 43

### 6-Benzyl-2-(5-difluoromethyl-furan-2-yl)-thieno[2,3-d]pyrimidin-4-ylamine

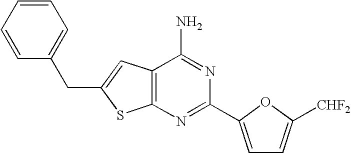

The title compound was prepared using 5-difluoromethyl-furan-2-carbonitrile in place of 5-methyl-furan-2-carbonitrile as described in Example 1. ¹H NMR (400 MHz, CDCl₃) δ=7.12-7.42 (m, 6H), 6.77-6.82 (m, 2H), 6.76 (t, J=54.4 Hz, 1H), 6.08 (br. s, 2H), 4.19 (s, 2H); MS m/e 358 (M+H).

### Example 44

### 6-Benzyl-2-(2-methoxy-pyridin-4-yl)-thieno[2,3-d]pyrimidin-4-ylamine

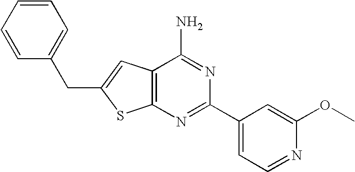

The title compound was prepared using 2-methoxy-isonicotinonitrile and in place of 5-methyl-furan-2-carbonitrile as described in Example 1. ¹H NMR (300 MHz, Acetone-d₆) δ=8.22 (d, J=5.3 Hz, 1H), 7.86 (d, J=5.3 Hz, 1H), 7.69 (s, 1H), 7.35 (d, J=4.5 Hz, 4H), 7.28 (s, 2H), 6.94 (br. s, 2H), 4.27 (s, 2H), 3.93 (s, 3H); MS m/e 349 (M+H).

### Example 45

### [4-Amino-2-(5-difluoromethyl-furan-2-yl)-thieno[2,3-d]pyrimidin-6-yl]-(2-methoxy-phenyl)-methanol

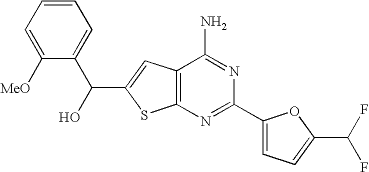

The title compound was prepared using 4-amino-2-(5-difluoromethyl-furan-2-yl)-thieno[2,3-d]pyrimidine-6-carbaldehyde (an intermediate prepared in Example 41) and 2-methoxyphenylmagnesium bromide in place of 4-amino-2-(5-methyl-furan-2-yl)-thieno[2,3-d]pyrimidine-6-carbaldehyde and phenylmagnesium bromide, respectively, as described in Example 2. ¹H NMR (300 MHz, Acetone-d₆) δ=7.61 (d, J=7.5 Hz, 1H), 7.23-7.37 (m, 2H), 7.18 (br. s, 1H), 6.99 (t, J=53.1 Hz, 1H), 6.85-7.07 (m, 5H), 6.39 (d, J=4.5 Hz, 1H), 5.29 (br. s, 1H), 3.85 (s, 3H); MS m/e 404 (M+H).

### Biological Assays and Activity

### Ligand Binding Assay for Adenosine A2a Receptor

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET1021). Assay was set up in 96-well polypropylene plate in total volume of 200 μL by sequentially adding 20 μL 1:20 diluted membrane, 130 μL assay buffer (50 mM Tris.HCl, pH7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris.HCl, pH7.4 containing 0.3% polyethylenimine. Plates were then washed 5 times with cold 50 mM Tris.HCl, pH7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

### Adenosine A2a Receptor Functional Assay (A2AGAL2)

To initiate the functional assay, cryopreserved CHO-K1 cells overexpressing the human adenosine A2a receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 uL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme colorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The colorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

### Adenosine A1 Receptor Functional Assay (A1GAL2)

To initiate the functional assay, cryopreserved CHO-K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 uL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

A2a ASSAY DATA

| Example | A2AGAL2 Ki μM | A2A-B Ki μM | A1GAL2 Ki μM |
|---|---|---|---|
| 1 | 0.0109926 | 0.0119399 | 0.27919 |
| 2 | 0.00227667 | ND | 0.0208161 |
| 3 | ND | ND | ND |
| 4 | ND | ND | ND |
| 5 | 0.230091 | 0.100092 | 0.50501 |
| 6 | ND | ND | ND |
| 7 | ND | 0.328322 | ND |
| 8 | 0.0487641 | ND | 0.222741 |
| 9 | 0.0309528 | ND | >0.593882 |
| 10 | 0.106758 | ND | >0.610098 |
| 11 | 0.0019829 | 0.00671583 | 0.0102117 |
| 12 | 0.0101251 | 0.0509918 | 0.115319 |
| 13 | 0.215328 | 0.192221 | 1.41775 |
| 14 | 0.0160731 | 0.0462914 | 1.49108 |
| 15 | 0.0132251 | ND | 0.185695 |
| 16 | 0.0478189 | ND | 0.909704 |
| 17 | 0.100161 | ND | 1.41579 |
| 18 | 0.304158 | ND | 3.16301 |
| 19 | 0.00248485 | ND | 0.100161 |
| 20 | 0.538766 | ND | 2.33185 |
| 21 | 0.0193776 | 0.0726775 | 1.17679 |
| 22 | 0.188539 | 0.0500034 | 2.7574 |
| 23 | 0.26339 | ND | 0.861391 |
| 24 | 0.0264302 | ND | >1.03825 |
| 25 | 0.0174944 | ND | 0.618301 |
| 26 | ND | ND | ND |
| 27 | ND | ND | ND |
| 28 | 0.0572268 | ND | 0.262603 |
| 29 | 0.0611646 | 0.00660998 | 1.57073 |
| 30 | 0.937994 | 0.172982 | 20.1511 |
| 31 | ND | ND | ND |
| 32 | 0.00241546 | ND | 0.0909494 |
| 33 | 0.235831 | 0.0260016 | 3.3381 |
| 34 | ND | ND | ND |
| 35 | ND | ND | ND |
| 36 | ND | ND | ND |
| 37 | ND | ND | ND |
| 38 | ND | ND | ND |
| 39 | 0.0130497 | ND | 0.515822 |
| 40 | ND | ND | ND |
| 41 | 0.0112538 | 0.0179019 | 0.223203 |
| 42 | ND | ND | ND |
| 43 | 0.0252348 | 0.0375059 | 2.99778 |
| 44 | ND | ND | ND |
| 45 | 0.00437019 | ND | 0.0343637 |

ND indicates that no data was available.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. A compound of Formula Z 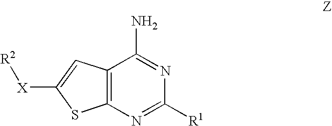
wherein:
X is selected from the group consisting of: 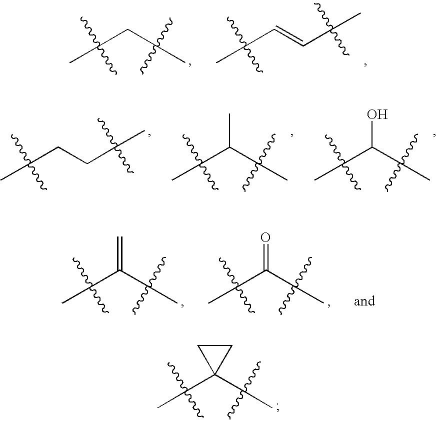
R¹ is heteroaryl optionally substituted with one substituent selected from the group consisting of: —OH, OC₍₁₋₄₎alkyl, CF₃, OCF₃, Cl, Br, —CN, F, CHF₂, C₍₁₋₄₎alkyl, and cyclopropyl;
R² is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN; wherein said C₍₁₋₄₎alkyl is optionally substituted with a ring selected from the group consisting of: 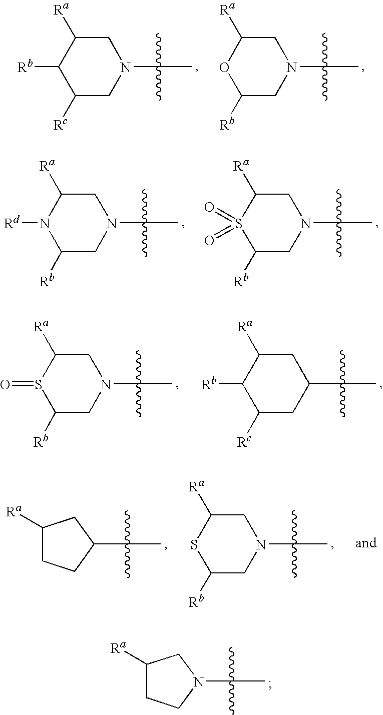
wherein R^{a}, R^{b}, and R^{C} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;
and solvates, hydrates, tautomers and pharmaceutically acceptable salts thereof.

2. A compound of claim 1, wherein:
R¹ is heteroaryl optionally substituted with one substituent selected from the group consisting of: —OH, OC₍₁₋₄₎alkyl, OCF₃, Cl, Br, —CN, F, CHF₂, C₍₁₋₄₎alkyl, and cyclopropyl;
R² is phenyl substituted with one substituent, selected from the group consisting of: H, —OH, OC₍₁₋₄₎alkyl, OCF₃, CHF₂, CF₃, Cl, Br, —CN, F, and C₍₁₋₄₎alkyl, wherein said C₍₁₋₄₎alkyl is optionally substituted with morpholinyl, piperidinyl, or piperazinyl;
and solvates, hydrates, tautomers and pharmaceutically acceptable salts thereof.

3. A compound of claim 2, wherein:
R¹ is heteroaryl selected from the group consisting of: furyl, thiazolyl, pyridyl, oxazolyl, imidazolyl, pyrimidyl, thiophenyl, and pyridazyl, wherein said heteroaryl is optionally substituted with one substituent selected from the group consisting of: OC₍₁₋₄₎alkyl, OCF₃, —CN, F, CHF₂, C₍₁₋₄₎alkyl, and cyclopropyl;
R² is phenyl substituted with one substituent, selected from the group consisting of: H, —OH, OC₍₁₋₄₎alkyl, OCF₃, CHF₂, CF₃, F, Cl, and C₍₁₋₄₎alkyl, wherein said C₍₁₋₄₎alkyl is optionally substituted with morpholinyl, piperidinyl, or piperazinyl;
and solvates, hydrates, tautomers and pharmaceutically acceptable salts thereof.

4. A compound of claim 3, wherein:
R¹ is heteroaryl selected from the group consisting of: furyl, thiazolyl, pyridyl, oxazolyl, imidazolyl, pyrimidyl, thiophenyl, and pyridazyl, wherein said heteroaryl is optionally substituted with one substituent selected from the group consisting of: C₍₁₋₄₎alkyl, OCH₃, —CN, CHF₂, and cyclopropyl;
R² is phenyl substituted with one substituent, selected from the group consisting of: H, —OH, OCH₃, OCF₃, CHF₂, CF₃, F, Cl, and C₍₁₋₄₎alkyl, wherein said C₍₁₋₄₎alkyl is optionally substituted with morpholinyl;
and solvates, hydrates, tautomers and pharmaceutically acceptable salts thereof.

5. A compound of claim 4, wherein:
R¹ is selected from the group consisting of: 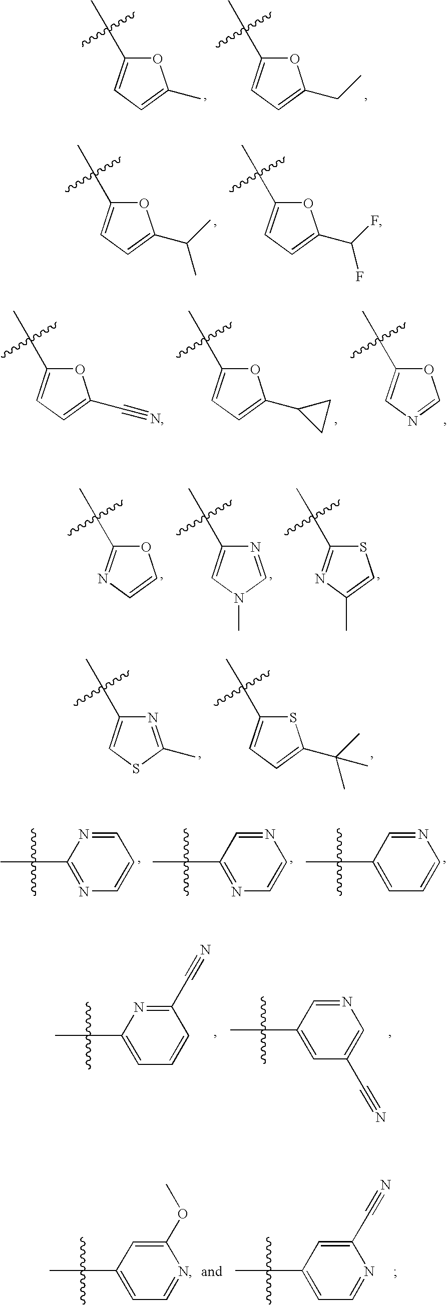
R² is phenyl substituted with one substituent, selected from the group consisting of: H, —CH₂-morpholinyl, OCH₃, F, and Cl;
and solvates, hydrates, tautomers and pharmaceutically acceptable salts thereof.

6. A compound selected from the group consisting of: 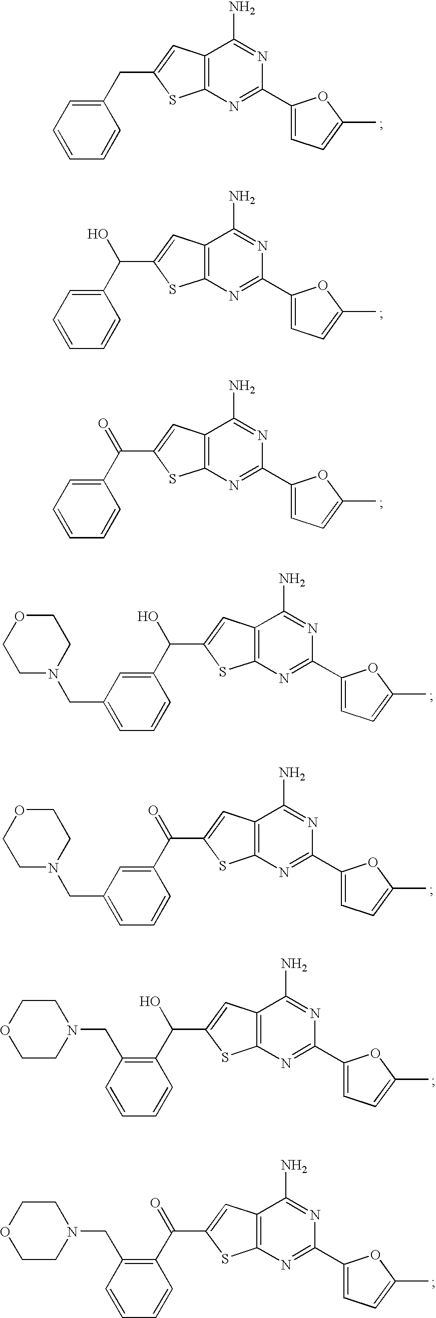 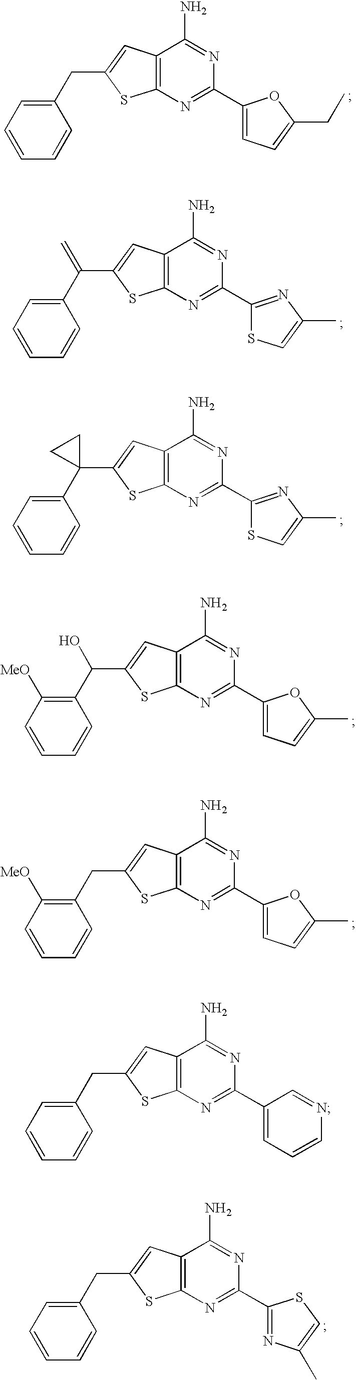 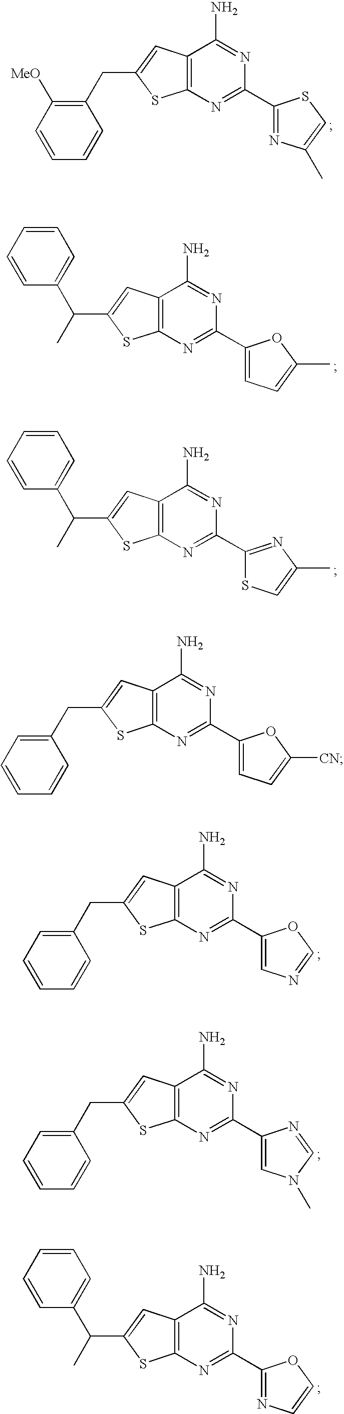 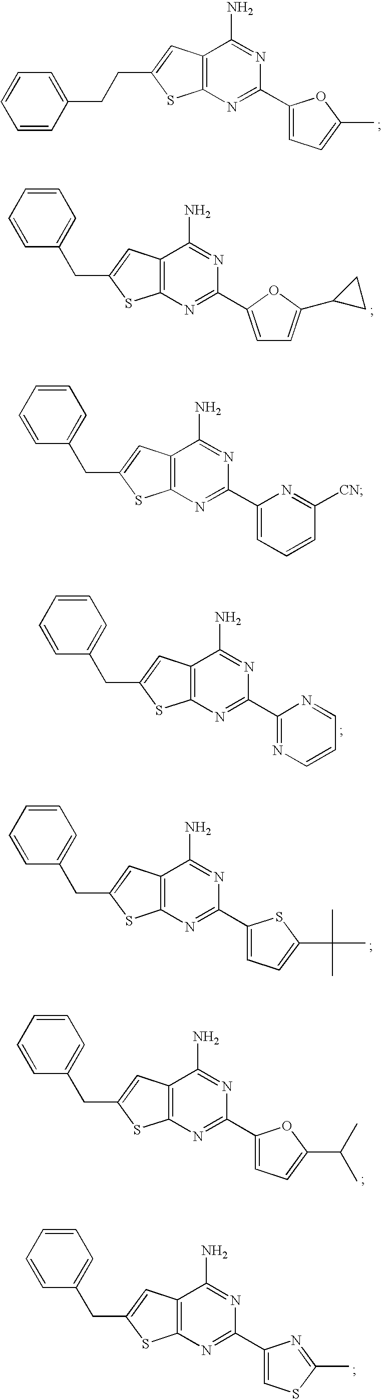 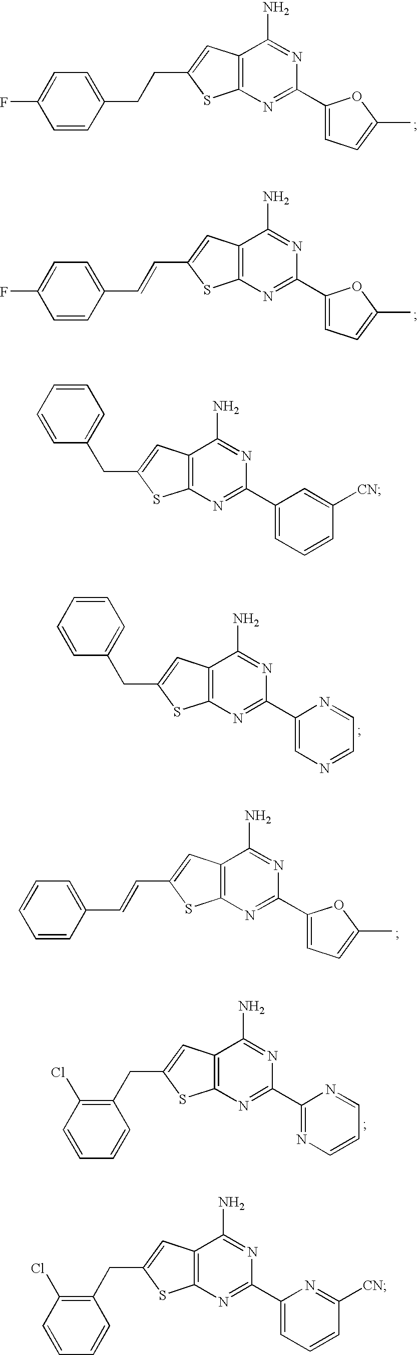 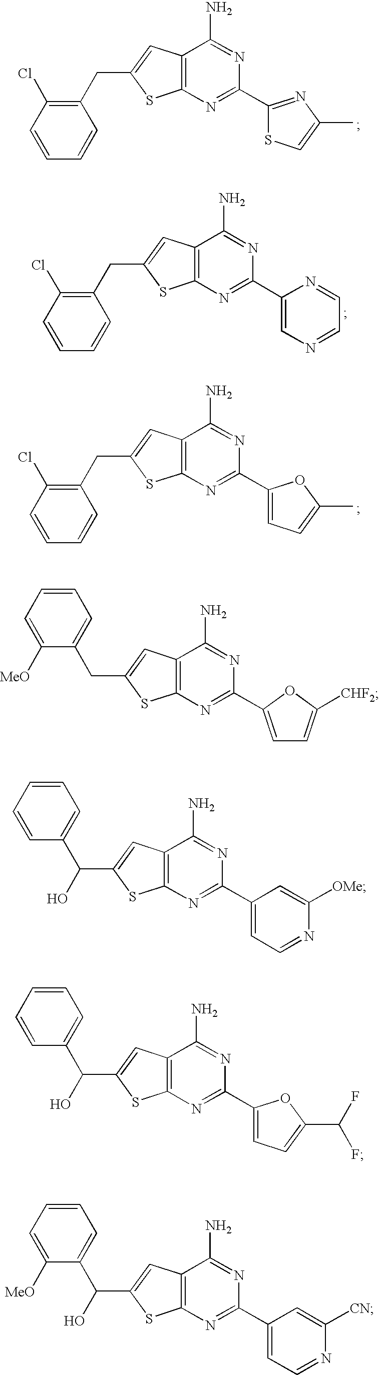 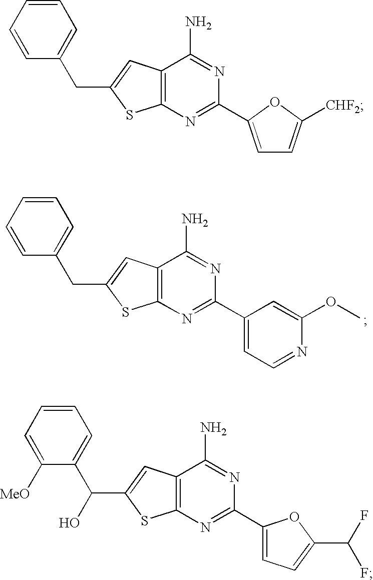
and solvates, hydrates, tautomers and pharmaceutically acceptable salts thereof.

7. A pharmaceutical composition comprising the compound of claim 1; and a pharmaceutically acceptable carrier.

8. A method of treating a subject having a disorder ameliorated by antagonizing Adenosine A2a receptors in appropriate cells in the subject, which comprises administering to the subject a therapeutically effective dose of the compound of claim 1.

9. A method of preventing a disorder ameliorated by antagonizing Adenosine A2a receptors in appropriate cells in the subject, comprising administering to the subject a prophylactically effective dose of the corn pound of claim 1 either preceding or subsequent to an event anticipated to cause a disorder ameliorated by antagonizing Adenosine A2a receptors in appropriate cells in the subject.

10. The method of claim 8 comprising administering to the subject a therapeutically or prophylactically effective dose of the pharmaceutical composition of claim 7.

11. The method of claim 9 comprising administering to the subject a therapeutically or prophylactically effective dose of the pharmaceutical composition of claim 7.

12. The method of claim 8, wherein the disorder is a neurodegenerative disorder or a movement disorder.

13. The method of claim 8, wherein the disorder is selected from the group consisting of Parkinson's Disease, Huntington's Disease, Multiple System Atrophy, Corticobasal Degeneration, Alzheimer's Disease, and Senile Dementia.

14. The method of claim 9, wherein the disorder is a neurodegenerative disorder or a movement disorder.

15. The method of claim 9, wherein the disorder is selected from the group consisting of Parkinson's Disease, Huntington's Disease, Multiple System Atrophy, Corticobasal Degeneration, Alzheimer's Disease, and Senile Dementia.

16. The method of claim 8, wherein the disorder is Parkinson's Disease.

17. The method of claim 8, wherein the disorder is addiction.

18. The method of claim 8, wherein the disorder is Attention Deficit Hyperactivity Disorder (ADHD).

19. The method of claim 8, wherein the disorder is depression.

20. The method of claim 8, wherein the disorder is anxiety.
